(19) United States
(12) Patent Application Publication
(10) Pub. No.: US 2013/0183350 A1
(43) Pub. Date: Jul. 18, 2013

(54) IMMUNOGENIC COMPOSITIONS

(71) Applicants: Kevin Harper, Cheltenham (CA); Belma Ljutic, Thornhill (CA); Scott Gallichan, Burlington (CA); Martina Ochs-Onolemhemhen, Lyon (FR); Garry Morefield, Nazareth, PA (US); Fernando Ausar, Markham (CA); Marie-Danielle Salha, Toronto (CA)

(72) Inventors: Kevin Harper, Cheltenham (CA); Belma Ljutic, Thornhill (CA); Scott Gallichan, Burlington (CA); Martina Ochs-Onolemhemhen, Lyon (FR); Garry Morefield, Nazareth, PA (US); Fernando Ausar, Markham (CA); Marie-Danielle Salha, Toronto (CA)

(21) Appl. No.: 13/515,093

(22) PCT Filed: Dec. 20, 2010
(86) PCT No.: PCT/CA2010/001977

§ 371 Date: Oct. 29, 2012

Related U.S. Application Data
(60) Provisional application No. 61/289,236, filed on Dec. 22, 2009.
(60) Provisional application No. 61/325,660, filed on Apr. 19, 2010.

(51) Int. Cl. A61K 39/09; C07K 16/12

(52) U.S. Cl. CPC A61K 39/092; C07K 16/1275
USPC 424/400; 424/244.1; 424/190.1; 424/139.1; 424/192.1

(57) ABSTRACT

This disclosure relates to immunogenic compositions comprising an isolated immunogenic S. pneumoniae PcpA polypeptide and at least one additional antigen (such as for example, an isolated immunogenic S. pneumoniae polypeptide selected from the group consisting of the polyhistidine triad family of proteins (e.g. PhtD) and methods of using these compositions for preventing and treating diseases caused by S. pneumoniae.

### CROSS-REFERENCE TO RELATED APPLICATIONS

The present applications claims priority to U.S. Ser. No. 61/289,236, filed Dec. 22, 2009; and U.S. Ser. No. 61/325,660, filed Apr. 19, 2010, which are incorporated by reference herein in their entireties.

### FIELD OF INVENTION

The present invention relates to the field of immunology and, in particular, to Streptococcus pneumoniae antigens and their use in immunization.

### BACKGROUND

Streptococcus pneumoniae is a rather ubiquitous human pathogen, frequently found in the upper respiratory tract of healthy children and adults. These bacteria can infect several organs including the lungs, the central nervous system (CNS), the middle ear, and the nasal tract and cause a range of diseases (i.e., symptomatic infections) such as for example, sinus infection, otitis media, bronchitis, pneumonia, meningitis, and bacteremia (septicemia). Pneumococcal meningitis, the most severe form of these pneumococcal diseases, is associated with significant mortality and morbidity despite antibiotic treatment (Quagliarello et. al. (1992) N. Engl. J. Med. 327:864-872). Children under the age of two and the elderly are particularly susceptible to symptomatic pneumococcal infections.

Currently, there are two available types of pneumococcal vaccines. The first includes capsular polysaccharides from 23 types of S. pneumoniae, which together represent the capsular types of about 90% of strains causing pneumococcal infection. This vaccine, however, is not very immunogenic in young children, an age group with heightened susceptibility to pneumococcal infection as they do not generate a good immune response to polysaccharide antigens prior to 2 years of age. In adults the vaccine has been shown to be about 60% efficacious against bacteremic pneumonia, but it is less efficacious in adults at higher risk of pneumococcal infection because of age or underlying medical conditions (Fedson, and Musher 2004, “Pneumococcal Polysaccharide Vaccine”, pp. 529-588; In Vaccines. S. A. Plotikin and W. A. Orenstein (eds.), W.B. Saunders and Co., Philadelphia, Pa.; Shapiro et. al., N. Engl. J. Med. 325:1453-1460 (1991)).

The second available type are conjugate vaccines. These vaccines which include serotype specific capsular polysaccharide antigens conjugated to a protein carrier, elicit serotype-specific protection (9). Currently available are 7-valent and 13-valent conjugate vaccines: the 7-valent includes 7 polysaccharide antigens (derived from the capsules of serotypes 4, 6B, 9V, 14, 18C, 19F and 23F) and the 13-valent includes 13 polysaccharide antigens (derived from the capsules of serotypes 1, 3, 5, 6A, 7F and 19A, in addition to those covered by the 7-valent). A 9-valent and 11-valent conjugate vaccine have also been developed and each includes polysaccharides specific for serotypes not covered by the 7-valent (i.e., serotypes 1 and 5 in the 9-valent and types 3 and 7F in the 11-valent).

The manufacture of conjugate vaccines is complex and costly due in part to the need to produce 7 (or 9 or 11) different polysaccharides each conjugated to the protein carrier. Such vaccines also do not do a good job of covering infections in the developing world where serotypes of Streptococcus pneumoniae not covered by the conjugate vaccines are very common (Di Fabio et al., Pediatr. Infect. Dis. J. 20:959-967 (2001); Mulholland, Trop. Med. Int. Health 10:497-500 (2005)). The use of the 7-valent conjugate vaccine has also been shown to have led to an increase in colonization and disease with strains of capsule types not represented by the 7 polysaccharides included in the vaccine (Bogaert et al., Lancet Infect. Dis. 4:144-154 (2004); Eskola et al., N. Engl. J. Med. 344-403-409 (2001); Mbelle et al., J. Infect. Dis. 180:1171-1176 (1999)).

As an alternative to the polysaccharide based vaccines currently available, a number of S. pneumoniae antigens have been suggested as possible candidates for a protein-based vaccine against S. pneumoniae. To date, however, no such vaccine is currently available on the market. Therefore, a need remains for effective treatments for S. pneumoniae.

### SUMMARY

Immunogenic compositions and methods for eliciting an immune response against Streptococcus infections (such as e.g., S. pneumoniae) are described. More particularly, the present disclosure relates to immunogenic compositions comprising immunogenic PcpA polypeptides and/or immunogenic polypeptides of the polyhistidine triad family (PhtX: PhtA, B, D, E), methods for their production and their use. Immunogenic PcpA and PhtX polypeptides (e.g. PhtD), including fragments of PcpA and PhtD and variants of each, and nucleic acids that encode the polypeptides are also provided. Immunogenic compositions comprising immunogenic PcpA polypeptides and/or immunogenic polypeptides of the polyhistidine triad family (PhtX: PhtA, B, D, E), and/or detoxified pneumolysin. Further provided, are methods of preparing antibodies against Streptococcus polypeptides and methods for treating and/or preventing Streptococcus infection (e.g., S. pneumoniae infection) using such antibodies.

Also provided are compositions, such as pharmaceutical compositions (e.g., vaccine compositions), including one or more immunogenic PcpA polypeptides, PhtX polypeptides and/or detoxified pneumolysin proteins. Optionally, the compositions can include an adjuvant. The compositions may also include one or more pharmaceutically acceptable excipients, which increase the thermal stability of the polypeptides/proteins relative to a composition lacking the one or more pharmaceutically acceptable excipients. In one example, the one or more pharmaceutically acceptable excipients increase the thermal stability of PcpA, PhtX and/or detoxified pneumolysin protein by 0.5° C. or more, relative to a composition lacking the one or more pharmaceutically acceptable excipients. The compositions can be in liquid form, dry powder form, freeze dried, spray dried and or foam dried. The one or more pharmaceutically acceptable excipients can be for example, selected from the group consisting of buffers, tonicity agents, simple carbohydrates, sugars, carbohydrate polymers, amino acids, oligopeptides, polyamino acids, polyhydric alcohols and ethers thereof, detergents, lipids, surfactants, antioxidants, salts, human serum albumin, gelatins, formaldehyde, or combinations thereof.

Also provided are methods of inducing an immune response to S. pneumoniae in a subject, which involve administering to the subject a composition as described herein. Use of the compositions of the invention in inducing an immune response to S. pneumoniae in a subject, or in preparation of medicaments for use in this purpose is also provided.

The invention provides several advantages. For example, administration of the compositions of the present invention to a subject elicits an immune response against infections by a number of strains of S. pneumoniae. In addition, the multivalent compositions of the present invention include specific combinations of immunogenic polypeptides of S. pneumoniae which when administered do not experience antigenic interference and may provide additive effects. Use of the excipients described herein can result in increased thermal stability of the polypeptides/proteins within the compositions.

Other features and advantages of the invention will be apparent from the following Detailed Description, the Drawings and the Claims.

### BRIEF DESCRIPTION OF FIGURES

The present invention will be further understood from the following description with reference to the drawings, in which:

FIG. 1 Depicts the serum anti-protein IgG antibody titres of mice immunized with varying doses of PcpA and PhtD (Example 2). In this study, recombinant PhtD and PcpA were combined with AlOOH adjuvant as monovalent or bivalent formulations. Balb/c mice were immunized subcutaneously 3 times at 3 weeks interval, and blood was collected prior to the first immunization and following the first, second and third immunizations. IgG titers were assessed by end-point ELISAs. All mice that had received PcpA and PhtD proteins generated antigen-specific antibody responses after immunization.

FIG. 2 a to d Depicts the serum anti-protein IgG antibody titres of rats immunized with 50 μg antigen/dose of PcpA and/or PhtD. In this study, rats were immunized on days 0, 21 and 42 with either a control of Tris Buffered Saline (10 mM Tris pH 7.4, 150 mM NaCl), aluminum hydroxide adjuvanted bivalent PhtD and PcpA, unadjuvanted bivalent PhtD and PcpA or aluminum hydroxide adjuvanted PcpA using 50 μg antigen/dose. Sera from pretest, day 44 and day 57 bleeds were tested for antibody titers to PhtD and PcpA specific IgG antibody titers by ELISA.

FIG. 3 Depicts the survival percentage for each group of mice immunized (Example 5). In this study, a bivalent formulation of recombinant PhtD and PcpA was evaluated using an intranasal challenge model. Immunized animals were challenged with a lethal dose of an S. pneumoniae strain (MD, 14453 or 941192).

FIG. 4a, 4b. FIG. 4a depicts the total antigen-specific IgG titres measured by endpoint dilution ELISA and geometric mean titres (+/−SD) for each group. FIG. 4b depicts total antigen-specific titres measured by quantitative ELISA. In this study (Example 7), bivalent compositions of PhtD and PcpA were prepared (using two different lots of each of PhtD and PcpA) and formulated with phosphate treated AlOOH (2 mM). Groups of 6 female CBA/j mice were immunized intramuscularly or subcutaneously three times at 3 week intervals with the applicable formulation. Mice were challenged a lethal dose of S. pneumoniae strain MD following the third (final) bleed.

FIG. 5 Depicts the survival percentage for each group. In this study (Example 6), bivalent compositions of PhtD and PcpA were prepared (using two different lots of each of PhtD and PcpA) and formulated with phosphate treated AlOOH (2 mM). Groups of 6 female CBA/j mice were immunized intramuscularly or subcutaneously three times at 3 week intervals with the applicable formulation. Mice were challenged a lethal dose of S. pneumoniae strain MD following the third bleed.

FIG. 6 Depicts Recognition of PcpA and PhtD on bacterial surface by Corresponding Rabbit Antisera on Various Pneumococcal Strains Grown in Mn2+ Depleted Media (Example 9).

FIG. 7 Depicts Binding of Purified Human Anti-PcpA and Anti-PhtD Antibodies to proteins (PcpA, PhtD) on bacterial cell surface of Strain WU2 (Example 9).

FIG. 8 Depicts % survival observed per log dilution of sera administered (Example 10).

FIG. 9 Depicts summary of the total IgG titers measured by ELISA (Example 11)

FIG. 10a to f The stability of PcpA and PhtD in monovalent and bivalent formulations (formulated with AlO(OH) or phosphate treated AlO(OH) (PTH). Formulations were prepared using AlO(OH) or PTH with a final concentration of 2 mM phosphate and then incubated at various temperatures (i.e., 5° C., 25° C., 37° C. or 45° C.). Intact antigen concentration was then assessed by RP-HPLC.

FIG. 11 Stability of PhtD and PcpA under stress conditions as evaluated by ELISA. Bivalent formulations at 100 μg/mL were incubated at 37° C. for 12 weeks and the antigenicity was evaluated by ELISA.

FIG. 12A Studies of excipient effects on the stability of PcpA (stored at 50° C. for three days) in the presence of 10% sorbitol (▪), 10% trehalose (), 10% sucrose (Δ), TBS pH 9.0 (♦), and TBS pH 7.4 (∘) by RP-HPLC.

FIG. 12B Studies of excipient effects on the antigenicity of PcpA (stored at 50° C. for three days) in the presence of 10% sorbitol, 10% trehalose, 10% sucrose, TBS pH 9.0, and TBS pH 7.4 by quantitative ELISA sandwich. Formulations were stored at 50° C. for three days. Antigenicity was evaluated for each formulation at time zero (white bars) and following three day storage (black bars).

FIG. 13 Effect of pH on the physical stability of adjuvanted proteins. PcpA (A), PhtD (B) and PlyD1 (C) were adjuvanted with aluminum hydroxide or aluminum phosphate at different pH values and the Tm values were obtained by derivative analysis of the fluorescence traces.

FIG. 14 Depicts the total antigen-specific IgG titres measured by endpoint dilution ELISA and geometric mean titres (+/−SD) for each group.

FIGS. 15 A, B, C Depicts the total antigen-specific IgG titres elicited as measured byT ELISA per antigen dose administered to mice.

### DETAILED DESCRIPTION OF INVENTION

Compositions and methods for eliciting an immune response against S. pneumoniae and for treating and preventing disease caused by S. pneumoniae in mammals, such as for example in humans are described. Provided are immunogenic compositions comprising immunogenic PcpA polypeptides and/or immunogenic polypeptides of the polyhistidine triad family (PhtX: PhtA, PhtB, PhtD, PhtE), methods for their production and their use. The compositions may include detoxified pneumolysin or immunogenic fragments thereof. Methods include passive and active immunization approaches, which include administration (e.g., subcutaneous, intramuscular) of immunogenic compositions comprising one or more substantially purified Streptococcal (e.g., S. pneumoniae) polypeptides, antibodies to the polypeptides themselves, or a combination thereof. The invention also includes Streptococcus sp. (e.g., S. pneumoniae) polypeptides, immunogenic compositions (e.g., vaccines) comprising Streptococcal polypeptides, methods of producing such compositions, and methods of producing Streptococcal (e.g., S. pneumoniae) antibodies. These methods and compositions are described further, below.

The compositions of the invention include one, two, three or more immunogenic polypeptides. The compositions may include for example, individually or in combination, an immunogenic polypeptide of PcpA; an immunogenic polypeptide of a member of the poly histidine triad family of proteins (e.g., PhtA, PhtB, PhtD, and PhtE, referenced herein as PhtX proteins); a detoxified pneumolysin polypeptide. Immunogenic fragments and fusions of these polypeptides may also be included in the compositions (e.g., a fusion of PhtB and PhtE). These immunogenic polypeptides may optionally be used in combination with pneumococcal saccharides or other pneumococcal polypeptides.

In one multi-component example, the immunogenic composition includes an immunogenic PcpA polypeptide and one or more immunogenic PhtX polypeptides. A preferred embodiment of such a composition comprises an immunogenic PhtD polypeptide and an immunogenic PcpA polypeptide. In another example, the composition includes an immunogenic PcpA polypeptide, an immunogenic PhtX polypeptide (e.g., PhtD) and detoxified pneumolysin. Certain embodiments of the immunogenic composition (in e.g., bivalent and trivalent form) are described in the Examples herein.

### Polypeptides

Immunogenic PcpA polypeptides comprise the full-length PcpA amino acid sequence (in the presence or absence of the signal sequence), fragments thereof, and variants thereof. PcpA polypeptides suitable for use in the compositions described herein include, for example, those of GenBank Accession No. CAB04758 from S. pneumoniae strain B6, GenBank Accession No. NP_from S. pneumoniae strain TIGR4 and GenBank Accession No. NP_{—}359536 from S. pneumoniae strain R6, and those from S. pneumoniae strain 14453.

The amino acid sequence of full length PcpA in the S. pneumoniae 14453 genome is SEQ ID NO. 2. Preferred PcpA polypeptides for use with the invention comprise an amino acid sequence having 50% or more identity (e.g., 60, 65, 70, 75. 80, 85, 85, 90, 91, 92, 93, 94, 95, 96, 97, 98, 99, 99.5% or more) to SEQ ID NO:2 or SEQ ID NO:7. Preferred polypeptides for use with the invention comprise a fragment of at least 8, 9, 10, 12, 14, 16, 18, 20, 25, 30, 35, 40, 50, 60, 70, 80, 90, 100, 150, 200, 250 or more consecutive amino acids of SEQ ID NO:2. Preferred fragments comprise an epitope from SEQ ID NO.2. Other preferred fragments lack one or more amino acids from the N-terminus of SEQ ID NO. 2 (e.g., 1, 2, 3, 4, 5, 6, 7, 8, 9, 10, 15, 20, 25 or more) and/or one or more amino acids from the C-terminus of SEQ ID NO:2 while retaining at least one epitope of SEQ ID NO:2. Further preferred fragments lack the signal sequence from the N-terminus of SEQ ID NO:2. A preferred PcpA polypeptide is SEQ ID NO:7.

Optionally, immunogenic polypeptides of PcpA comprise one or more leucine rich regions (LRRs). These LLRs are present in naturally occurring PcpA or have about 60 to about 99% sequence identity, including, for example, 80%, 85%, 90% or 95% sequence identity to the naturally occurring LRRs. LRRs in the mature PcpA protein (i.e., the protein lacking the signal peptide) can be found in certain sequences disclosed in WO 2008/022302 (e.g., SEQ ID NOs:1, 2, 41 and 45 of WO 2008/022302).

An immunogenic polypeptide of PcpA optionally lacks the choline binding domain anchor sequence typically present in the naturally occurring mature PcpA protein. The naturally occurring sequence of the choline binding anchor of the mature PcpA protein is disclosed in WO 2008/022302 as SEQ ID NO:52. More particularly, an immunogenic polypeptide comprises an N-terminal region of naturally occurring PcpA with one or more amino acid substitutions and about 60 to about 99% sequence identity or any identity in between, e.g. 80, 85, 90 and 95% identity, to the naturally occurring PcpA. The N-terminal region may comprise the amino acid sequence of SEQ ID NO: 2 (or SEQ ID NOs: 1, 2, 3, 4, 41 or 45 of WO2008/022302), in the presence or absence of one or more conservative amino acid substitutions and in the presence or absence of the signal sequence. The N-terminal region may comprise an amino acid sequence having about 60 to about 99% sequence identity (or any identity in between 80 to 99% identity) to SEQ ID NOs: 1 or 7 (set out in the Sequence Listing herein) or SEQ ID NOs:1, 2, 3, 4, or 41 of WO2008/022302.

Immunogenic fragments of SEQ ID NOs: 2 and 7 comprise 5, 10, 20, 30, 40, 50, 60, 70, 80, 90, 100, 110, 120, 130, 140, 150, 160, 170, 180, 190 and 191 amino acid residues of SEQ ID NOs: 2 and 7 or any number of amino acid residues between 5 and 191. Examples of immunogenic fragments of PcpA are disclosed in WO 2008/022302.

Optionally, immunogenic polypeptides of PcpA lack the LRRs. Examples of immunogenic polypeptides lacking the LRR are disclosed in WO 2008/022302 as SEQ ID NO:29, SEQ ID NO:30, and SEQ ID NO:31.

Immunogenic PhtX polypeptides suitable for the compositions of the invention comprise the full-length PhtA, PhtB, PhtD or PhtE amino acid sequence (in the presence or absence of the signal sequence), immunogenic fragments thereof, variants thereof and fusion proteins thereof. PhtD polypeptides suitable for use in the compositions described herein include, for example, those of GenBank Accession Nos. AAK06760, YP816370 and NP35851, among others. The amino acid sequence of full length PhtD in the S. pneumoniae 14453 genome is SEQ ID NO:1. A preferred polypeptide of PhtD (derived from the S. pneumoniae 14453 genome) is SEQ ID NO:5.

The immunogenic fragments of PhtX polypeptides of the present invention are capable of eliciting an immune response specific for the corresponding full length mature amino acid sequence.

Immunogenic PhtX (e.g., PhtD) polypeptides include the full length protein with the signal sequence attached, the mature full length protein with the signal peptide (e.g., 20 amino acids at N-terminus) removed, variants of PhtX (naturally occurring or otherwise, e.g., synthetically derived) and immunogenic fragments of PhtX (e.g., fragments comprising at least 15 or 20 contiguous amino acids present in the naturally occurring mature PhtX protein).

Examples of immunogenic fragments of PhtD are disclosed in PCT publication WO2009/012588.

Preferred PhtD polypeptides for use with the invention comprise an amino acid sequence having 50% or more identity (e.g., 60, 65, 70, 75. 80, 85, 85, 90, 91, 92, 93, 94, 95, 96, 97, 98, 99, 99.5% or more) to SEQ ID NO:1 or to SEQ ID NO:5. Preferred polypeptides for use with the invention comprise a fragment of at least 8, 9, 10, 12, 14, 16, 18, 20, 25, 30, 35, 40, 50, 60, 70, 80, 90, 100, 150, 200, 250 or more consecutive amino acids of SEQ ID NO:1. Preferred fragments comprise an epitope from SEQ ID NO.1 or to SEQ ID NO:5. Other preferred fragments lack one or more amino acids from the N-terminus of SEQ ID NO. 1 (e.g., 1, 2, 3, 4, 5, 6, 7, 8, 9, 10, 15, 20, 25 or more) and/or one or amino acids from the C-terminus of SEQ ID NO:1 while retaining at least one epitope of SEQ ID NO:1. Further preferred fragments lack the signal sequence from the N-terminus of SEQ ID NO:1. A preferred PhtD polypeptide is SEQ ID NO:5.

Pneumolysin (Ply) is a cytolytic-activating toxin implicated in multiple steps of pneumococcal pathogenesis, including the inhibition of ciliary beating and the disruption of tight junctions between epithelial cells (Hirst et al. Clinical and Experimental Immunology (2004)). Several pneumolysins are known and (following detoxification) would be suitable for use in the compositions described herein including, for example GenBank Accession Nos. Q04IN8, P0C2J9, Q7ZAK5, and ABO21381, among others. In one embodiment, Ply has the amino acid sequence shown in SEQ ID NO.10.

Immunogenic pneumolysin polypeptides for use with the invention include the full length protein with the signal sequence attached, the mature full length protein with the signal peptide removed, variants of pneumolysin (naturally occurring or otherwise, e.g., synthetically derived) and immunogenic fragments of pneumolysin (e.g., fragments comprising at least 15 or 20 contiguous amino acids present in the naturally occurring mature pneumolysin protein).

Immunogenic variants and fragments of the immunogenic pneumolysin polypeptides of the present invention are capable of eliciting an immune response specific for the corresponding full length mature amino acid sequence. The immunogenic pneumolysin polypeptides of the present invention are detoxified; that is, they lack or have reduced toxicity as compared to the mature wild-type pneumolysin protein produced and released by S. pneumoniae. The immunogenic pneumolysin polypeptides of the present invention may be detoxified for example, chemically (e.g., using formaldehyde treatment) or genetically (e.g., recombinantly produced in a mutated form).

Preferred examples of the immunogenic detoxified pneumolysin for use in the present invention are disclosed in PCT Publication No. WO 2010/071986. As disclosed in that application, the detoxified pneumolysin may be a mutant pneumolysin protein comprising amino acid substitutions at positions 65, 293 and 428 of the wild type sequence. In a preferred detoxified pneumolysin protein, the three amino acid substitutions comprise T₆₅→C, G₂₉₃→C, and C₄₂₈→A. A preferred immunogenic and detoxified pneumolysin polypeptide is SEQ ID NO:9.

Preferred pneumolysin polypeptides for use with the invention comprise an amino acid sequence having 50% or more identity (e.g., 60, 65, 70, 75, 80, 85, 85, 90, 91, 92, 93, 94, 95, 96, 97, 98, 99, 99.5% or more) to SEQ ID NO:9 or to SEQ ID NO:10. Preferred polypeptides for use with the invention comprise a fragment of at least 8, 9, 10, 12, 14, 16, 18, 20, 25, 30, 35, 40, 50, 60, 70, 80, 90, 100, 150, 200, 250 or more consecutive amino acids of SEQ ID NO:9 or 10. Preferred fragments comprise an epitope from SEQ ID NO.9 or to SEQ ID NO:10. Other preferred fragments lack one or more amino acids from the N-terminus of SEQ ID NO. 9 or 10 (e.g., 1, 2, 3, 4, 5, 6, 7, 8, 9, 10, 15, 20, 25 or more) and/or one or amino acids from the C-terminus of SEQ ID NO:9 or 10 while retaining at least one epitope of SEQ ID NO:9 or 10. Further preferred fragments lack the signal sequence from the N-terminus of SEQ ID NO:10.

The immunogenic polypeptides of PcpA, PhtX (e.g., PhtD), and pneumolysin described herein, and fragments thereof, include variants. Such variants of the immunogenic polypeptides described herein are selected for their immunogenic capacity using methods well known in the art and may comprise one or more conservative amino acid modifications. Variants of the immunogenic polypeptides (of PcpA, PhtD, pneumolysin) include amino acid sequence having about 60 to about 99% sequence identity (or any identity in between 60 and 99% identity) to the disclosed sequences (i.e., SEQ ID NO:2 or 7 (PcpA); SEQ ID NO:1 or 5 (PhtD); SEQ ID NO: 9 or 10 (Ply)). Amino acid sequence modifications include substitutional, insertional or deletional changes. Substitutions, deletions, insertions or any combination thereof may be combined in a single variant so long as the variant is an immunogenic polypeptide. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in a recombinant cell culture. Techniques for making substitution mutations are predetermined sites in DNA having a known sequence are well known and include, but are not limited to, M13 primer mutagenesis and PCR mutagenesis. Amino acid substitutions are typically of single residues but can occur at a number of different locations at once. Substitutional variants are those in which at least one residue has been removed and a different residue inserted in its place. Such substitutions generally are made in accordance with the following Table and are referred to as conservative substitutions. Others are well known to those of skill in the art.

As used herein, the amino acid substitution may be conservative or non-conservative. Conservative amino acid substitutions may involve a substitution of a native amino acid residue with a non-native residue such that there is little or no effect on the size, polarity, charge, hydrophobicity, or hydrophilicity of the amino acid residue at that position and, in particular, does not result in decreased immunogenicity. Suitable conservative amino acid substitutions are shown in the Table 1 below.

TABLE 1

| | | Preferred |
|---|---|---|
| Original | | Conservative |
| Residues | Exemplary Conservative Substitutions | Substitution |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

The specific amino acid substitution selected may depend on the location of the site selected. In certain embodiments, nucleotides encoding polypeptides and/or fragments are substituted based on the degeneracy of the genetic code (i.e., consistent with the “Wobble” hypothesis). Where the nucleic acid is a recombinant DNA molecule useful for expressing a polypeptide in a cell (e.g., an expression vector), a Wobble-type substitution will result in the expression of a polypeptide with the same amino acid sequence as that originally encoded by the DNA molecule. As described above, however, substitutions may be conservative, or non-conservative, or any combination thereof. A skilled artisan will be able to determine suitable variants of the polypeptides and/or fragments provided herein using well-known techniques.

Analogs can differ from naturally occurring S. pneumoniae polypeptides in amino acid sequence and/or by virtue of non-sequence modifications. Non-sequence modifications include changes in acetylation, methylation, phosphorylation, carboxylation, or glycosylation. A “modification” of a polypeptide of the present invention includes polypeptides (or analogs thereof, such as, e.g. fragments thereof) that are chemically or enzymatically derived at one or more constituent amino acid. Such modifications can include, for example, side chain modifications, backbone modifications, and N- and C-terminal modifications such as, for example, acetylation, hydroxylation, methylation, amidation, and the attachment of carbonhydrate or lipid moieties, cofactors, and the like, and combinations thereof. Modified polypeptides of the invention may retain the biological activity of the unmodified polypeptides or may exhibit a reduced or increased biological activity.

Structural similarity of two polypeptides can be determined by aligning the residues of the two polypeptides (for example, a candidate polypeptide and the polypeptide of, for example, SEQ ID NO: 2) to optimize the number of identical amino acids along the length of their sequences; gaps in either or both sequences are permitted in making the alignment in order to optimize the number of identical amino acids, although the amino acids in each sequence must nonetheless remain in their proper order. A candidate polypeptide is the polypeptide being compared to the reference polypeptide. A candidate polypeptide can be isolated, for example, from a microbe, or can be produced using a recombinant techniques, or chemically or enzymatically synthesized.

A pair-wise comparison analysis of amino acids sequences can be carried out using a global algorithm, for example, Needleman-Wunsch. Alternatively, polypeptides may be compared using a local alignment algorithm such as the Blastp program of the BLAST 2 search algorithm, as described by Tatiana et al., (FEMS Microbiol. Lett, 174 247-250 (1999), and available on the National Centre for Biotechnology Information (NCBI) website. The default values for all BLAST 2 search parameters may be used, including matrix=BLOSUM62; open gap penalty=11, extension gap penalty=1, gap×dropoff=50, expect 10, wordsize=3, and filter on. The Smith and Waterman algorithm is another local alignment tool that can be used (1988).

In the comparison of two amino acid sequences, structural similarly may be referred to by percent “identity” or may be referred to by percent “similarity.” “Identity” refers to the presence of identical amino acids. “Similarity” refers to the presence of not only identical amino acid but also the presence of conservative substitutions. A conservative substitution for an amino acid in a polypeptide of the invention may be selected from other members of the class to which the amino acid belongs, shown on Table 1.

The nucleic acids encoding the immunogenic polypeptides may be isolated for example, but without limitation from wild type or mutant S. pneumoniae cells or alternatively, may be obtained directly from the DNA of an S. pneumoniae strain carrying the applicable DNA gene (e.g., pcpA, phtD, ply), by using the polymerase chain reaction (PCR) or by using alternative standard techniques that are recognized by one skilled in the art. Possible strains of use include for example, S. pneumoniae strains TIGR4 and 14453. In preferred embodiments the polypeptides are recombinantly derived from S. pneumoniae strain 14453. Preferred examples of the isolated nucleic acid molecules of the present invention have nucleic acid sequences set out in SEQ ID NOs: 3, 4, 6 and 8. Sequence-conservative variants and function-conservative variants of these sequences are encompassed by the present invention.

The polypeptides of the present invention can be produced using standard molecular biology techniques and expression systems (see for example, Molecular Cloning: A Laboratory Manual, Third Edition by Sambrook et. al., Cold Spring Harbor Press, 2001). For example, a fragment of a gene that encodes an immunogenic polypeptide may be isolated and the polynucleotide encoding the immunogenic polypeptide may be cloned into any commercially available expression vector (such as, e.g., pBR322, and pUC vectors (New England Biolabs, Inc., Ipswich, Mass.)) or expression/purification vectors (such as e.g., GST fusion vectors (Pfizer, Inc., Piscataway, N.J.)) and then expressed in a suitable prokaryotic, viral or eukaryotic host. Purification may then be achieved by conventional means, or in the case of a commercial expression/purification system, in accordance with manufacturer's instructions.

Alternatively, the immunogenic polypeptides of the present invention, including variants, may be isolated for example, but without limitation, from wild-type or mutant S. pneumoniae cells, and through chemical synthesization using commercially automated procedures, such as for example, exclusive solid phase synthesis, partial solid phase methods, fragment condensation or solution synthesis.

Polypeptides of the present invention preferably have immunogenic activity. “Immunogenic activity” refers to the ability of a polypeptide to elicit an immunological response in a subject. An immunological response to a polypeptide is the development in a subject of a cellular and/or antibody-mediated immune response to the polypeptide. Usually, an immunological response includes but is not limited to one or more of the following effects: the product of antibodies, B cells, helper T cells, suppressor T cells and/or cytotoxic T cells, directed to an epitope or epitopes of the polypeptide. The term “Epitope” refers to the site on an antigen to which specific B cells and/or T cells respond so that antibody is produced. The immunogenic activity may be protective. The term “Protective immunogenic activity” refers to the ability of a polypeptide to elicit an immunological response in a subject that prevents or inhibits infection by S. pneumoniae (resulting in disease).

### Compositions

The disclosed immunogenic S. pneumoniae polypeptides are used to produce immunogenic compositions such as, for example, vaccine compositions. An immunogenic composition is one that, upon administration to a subject (e.g., a mammal), induces or enhances an immune response directed against the antigen contained within the composition. This response may include the generation of antibodies (e.g., through the stimulation of B cells) or a T cell-based response (e.g., a cytolytic response). These responses may or may not be protective or neutralizing. A protective or neutralizing immune response is one that is detrimental to the infectious organism corresponding to the antigen (e.g., from which the antigen was derived) and beneficial to the subject (e.g., by reducing or preventing infection). As used herein, protective or neutralizing antibodies may be reactive to the corresponding wild-type S. pneumoniae polypeptide (or fragment thereof) and reduce or inhibit the lethality of the corresponding wild-type S. pneumoniae polypeptide when tested in animals. An immunogenic composition that, upon administration to a host, results in a protective or neutralizing immune response may be considered a vaccine.

The compositions include immunogenic polypeptides in amounts sufficient to elicit an immune response when administered to a subject. Immunogenic compositions used as vaccines comprise an immunogenic polypeptide in an immunologically effective amount, as well as any other components, as needed. By ‘immunologically effective amount’, it is meant that the administration of that amount to a subject, either in a single dose or as part of a series, is effective for treatment or prevention.

In compositions that are comprised of two, three or more immunogenic polypeptides (e.g., PcpA, PhtD, and/or detoxified pneumolysin), the polypeptide components are preferably compatible and are combined in appropriate ratios to avoid antigenic interference and to optimize any possible synergies. For example, the amounts of each component can be in the range of about 5 μg to about 500 μg per dose, 5 μg to about 100 μg per dose; or 25 μg to about 50 μg per dose. Preferably the range can be 5 or 6 μg to 50 μg per antigenic component per dose. In one example, a composition includes 25 μg of an immunogenic polypeptide of PhtX (e.g., PhtD) and 25 μg of an immunogenic polypeptide of PcpA. The composition, in a different example, also includes 25 μg of pneumolysin (e.g. detoxified pneumolysin; PlyD1 (SEQ ID NO:9).

In the Examples set out below, in animal models, various antigen ratios were compared for a two-component vaccine composition of PhtX (e.g., PhtD) and PcpA, and for a three-component vaccine composition of PcpA, PhtX (e.g., PhtD) and detoxified pneumolysin (e.g., PlyD1). Surprisingly, statistically significant antigenic interference was not observed at the antigen ratios tested. Also, surprisingly antigen-specific antibodies elicited in response to immunization with the bivalent composition (or trivalent composition) were found to act in an additive manner in a passive immunization study in mice using rabbit sera. Thus, in a multi-component composition these components may be present in equivalent amounts (e.g. 1:1, 1:1:1). The components may be present in other ratios having regard to the estimated minimum antigen dose for each antigen (e.g., PcpA:PhtX(PhtD):Pneumolysin, about 1:1:1 to about 1:5:25). In one example, a trivalent composition comprises PcpA, PhtD and pneumolysin (e.g. PlyD1) in amounts (μg/dose) at a ratio of PcpA:PhtD:pneumolysin of 1:4:8. In a different example, the ratio of PcpA:PhtD:pneumolysin is 1:1:1.

Compositions of the invention can be administered by an appropriate route such as for example, percutaneous (e.g., intramuscular, intravenous, intraperitoneal or subcutaneous), transdermal, mucosal (e.g., intranasal) or topical, in amounts and in regimes determined to be appropriate by those skilled in the art. For example, 1-250 μg or 10-100 μg of the composition can be administered. For the purposes of prophylaxis or therapy, the composition can be administered 1, 2, 3, 4 or more times. In one example, the one or more administrations may occur as part of a “prime-boost” protocol. When multiple doses are administered, the doses can be separated from one another by, for example, one week, one month or several months.

Compositions (e.g., vaccine compositions) of the present invention may be administered in the presence or absence of an adjuvant. Adjuvants generally are substances that can enhance the immunogenicity of antigens. Adjuvants may play a role in both acquired and innate immunity (e.g., toll-like receptors) and may function in a variety of ways, not all of which are understood.

Many substances, both natural and synthetic, have been shown to function as adjuvants. For example, adjuvants may include, but are not limited to, mineral salts, squalene mixtures, muramyl peptide, saponin derivatives, mycobacterium cell wall preparations, certain emulsions, monophosphoryl lipid A, mycolic acid derivatives, nonionic block copolymer surfactants, Quil A, cholera toxin B subunit, polyphosphazene and derivatives, immunostimulating complexes (ISCOMs), cytokine adjuvants, MF59 adjuvant, lipid adjuvants, mucosal adjuvants, certain bacterial exotoxins and other components, certain oligonucleotides, PLG, and others. These adjuvants may be used in the compositions and methods described herein.

In certain embodiments, the composition is administered in the presence of an adjuvant that comprises an oil-in-water emulsion comprising at least squalene, an aqueous solvent, a polyoxyethylene alkyl ether hydrophilic nonionic surfactant, a hydrophobic nonionic surfactant, wherein said oil-in-water emulsion is obtainable by a phase inversion temperature process and wherein 90% of the population by volume of the oil drops has a size less than 200 nm, and optionally less than 150 nm. Such an adjuvant is described in WO2007006939 (Vaccine Composition Comprising a Thermoinversable Emulsion) which is incorporate herein in its entirety. The composition may also include the product E6020 (having CAS Number 287180-63-6), in addition to, or instead of the described squalene oil-in-water emulsion. Product E6020 is described in US2007/0082875 (which is incorporated herein by reference in its entirety).

In certain embodiments, the composition includes a TLR agonist (e.g., TLR4 agonist) alone or together in combination with an adjuvant. For example, the adjuvant may comprise a TLR4 agonist (e.g., TLA4), squalene, an aqueous solvent, a nonionic hydrophilic surfactant belonging to the polyoxyethylene alkyl ether chemical group, a nonionic hydrophobic surfactant and which is thermoreversible. Examples of such adjuvants are described in WO2007080308 (Thermoreversible Oil-in-Water Emulsion) which is incorporated herein in its entirety. In one embodiment, the composition is adjuvanted with a combination of CpG and an aluminum salt adjuvant (e.g., Alum).

Aluminum salt adjuvants (or compounds) are among the adjuvants of use in the practice of the invention. Examples of aluminum salt adjuvants of use include aluminum hydroxide (e.g., crystalline aluminum oxyhydroxide AlO(OH), and aluminum hydroxide Al(OH)₃. Aluminum hydroxide is an aluminum compound comprising Al³⁺ ions and hydroxyl groups (—OH). Mixtures of aluminum hydroxide with other aluminum compounds (e.g., hydroxyphosphate or hydroxy sulfate) may also be of use where the resulting mixture is an aluminum compound comprising hydroxyl groups. In particular embodiments, the aluminum adjuvant is aluminum oxyhydroxide (e.g., Alhydrogel®). It is well known in the art that compositions with aluminum salt adjuvants should not be exposed to extreme temperatures, i.e. below freezing (0° C.) or extreme heat (e.g., ≧70° C.) as such exposure may adversely affect the stability and the immunogenicity of both the adsorbed antigen and adjuvant.

The inventors have noted that the degradation rate of PcpA and PhtD polypeptides when adjuvanted with aluminum hydroxide adjuvant (AlO(OH)) is high (as discussed in the examples below). The inventors have found that adjuvanting PcpA and PhtD polypeptides with an aluminum compound comprising hydroxide groups (e.g., aluminum hydroxide adjuvant) that has been pretreated with phosphate, carbonate, sulfate, carboxylate, diphosphonate or a mixture of two or more of these compounds, increases the stability of these polypeptides. Thus, provided herein are formulations of compositions comprising an immunogenic PcpA polypeptide or an immunogenic PhtX polypeptide (e.g., PhtD) and an aluminum compound comprising hydroxide groups that has been treated with phosphate, carbonate, sulfate, carboxylate, diphosphonate or a mixture of two or more of these compounds, where the treatment increases the stability of the immunogenic polypeptide relative to a composition where the polypeptide is adsorbed to an untreated aluminum compound. In preferred embodiments the aluminum compound is treated with phosphate. Multivalent compositions comprising both immunogenic polypeptides of PcpA and PhtX (e.g., PhtD) and an aluminum compound comprising hydroxide groups that has been treated with phosphate, carbonate, sulfate, carboxylate, diphosphonate or a mixture of two or more of these compounds, where the treatment increases the stability of the immunogenic polypeptides relative to a composition where the polypeptide is adsorbed to an untreated aluminum compound are also provided.

In a particular embodiment of the invention, the aluminum compound (e.g., aluminum hydroxide adjuvant) is treated with phosphate, carbonate, sulfate, carboxylate, diphosphonate, or a mixture of two or more of these compounds. By treating the aluminum compound in this way a number of the hydroxyl groups (—OH) in the aluminum compound are replaced with the corresponding ion with which it is being treated (e.g., phosphate (PO₄)). This replacement lowers the PZC of the aluminum compound and the pH of the compound's microenvironment. The phosphate, carbonate, sulfate, carboxylate, or diphosphonate ions are added in an amount sufficient to lower the pH of the microenvironment to a level at which the antigen is stabilized (i.e., the rate of antigen hydrolysis is decreased). The amount necessary will depend on a number of factors such as, for example, the antigen involved, the antigen's isoelectric point, the antigen's concentration, the adjuvanting method utilized, and the amount and nature of any additional antigens present in the formulation. Those skilled in the art in the field of vaccines are capable of assessing the relevant factors and determining the concentration of phosphate, carbonate, sulfate, carboxylate, diphosphonate to add to the aluminum compound to increase the stability of the antigen (and therefore, can prepare the corresponding formulation and composition). For example, titration studies (i.e., adding increasing concentrations of phosphate, etc., to aluminum compound) may be performed.

Phosphate compounds suitable for use include any of the chemical compounds related to phosphoric acid (such as for example, inorganic salts and organic esters of phosphoric acid). Phosphate salts are inorganic compounds containing the phosphate ion (PO₄³⁻), the hydrogen phosphate ion (HPO₄²⁻) or the dihydrogen phosphate ion (H₂PO⁴⁻) along with any cation. Phosphate esters are organic compounds in which the hydrogens of phosphoric acid are replaced by organic groups. Examples of compounds that may be used in place of phosphate salts include anionic amino acids (e.g., glutamate, aspartate) and phospholipids.

Carboxylate compounds suitable for use include any of the organic esters, salts and anions of carboxylic acids (e.g., malic acid, lactic acid, fumaric acid, glutaric acid, EDTA, and EGTA). Sulfer anions suitable for use include any compound containing the sulfate (SO₄ radical) such as salts or esters of sulfuric acid (e.g., sodium sulfate, ammonium sulfate, sulfite, metabisulfite, thiosulfate). Examples of disphosphonate compounds suitable for use include clodronate, pamidronate, tiludronate, and alendronate.

In a preferred embodiment of the invention, phosphate is added to aluminum hydroxide adjuvant in the form of a salt. Preferably, the phosphate ions are provided by a buffer solution comprising disodium monosodium phosphate.

In the preferred practice of the present invention, as exemplified herein, the aluminum compound (e.g., aluminum oxyhydroxide) is treated with phosphate (for example, by a process as described in the examples). In this process, an aqueous suspension of aluminum oxyhydroxide (approximately 20 mg/mL) is mixed with a phosphate buffer solution (e.g., approximately 400 mol/L). The preferable final phosphate concentration is from about 2 mM to 20 mM. The mixture is then diluted with a buffer (e.g., Tris-HCl, Tris-HCl with saline HEPES) to prepare a suspension of aluminum oxyhydroxide and phosphate (PO₄). Preferably the buffer is 10 mM Tris-HCl and 150 mM NaCl at a pH of about 7.4. The suspension is then mixed for approximately 24 hr at room temperature. Preferably the concentration of elemental aluminum in the final suspension is within a range from about 0.28 mg/mL to 1.68 mg/mL. More preferably, the concentration of elemental aluminum is about 0.56 mg/mL.

Immunogenic polypeptides of PcpA, PhtD and detoxified pneumolysin (individually or in combination) may then be adsorbed to the treated aluminum hydroxide. Preferably, approximately 0.2-0.4 mg/mL of antigen is mixed with the suspension of treated aluminum hydroxide adjuvant (e.g., at room temperature or at 2-8° C., in an orbital mixer, for approximately 30 min, or approximately 12-15 hours, or approximately 24 hours).

The percentage of antigen adsorption may be assessed using standard methods known in the art. For example, an aliquot of the antigen/adjuvant preparation may be removed and centrifuged (e.g., at 10,000 rpm) to separate the unadsorbed protein (pellet) from the adjuvant suspension (supernatant). The concentration of protein in the supernatant may be determined using the bicinchoninic acid protein assay (BCA) or reverse phase-high performance liquid chromatography (RP-HPLC). The percentage of adsorption is calculated as follows: % A=100−([PrSN]×100/[PrCtr]) where, [PrSN] is the concentration of protein in supernatant and [PfCtr] is the concentration in the corresponding unadjuvanted control. In preferred embodiments, the % adsorption ranges from about 70% to about 100%. In more preferred embodiments the % adsorption is at least about 70%.

In one embodiment of adjuvanted immunization, immunogenic polypeptides and/or fragments thereof may be covalently coupled to bacterial polysaccharides to form polysaccharide conjugates. Such conjugates may be useful as immunogens for eliciting a T cell dependent immunogenic response directed against the bacterial polysaccharide conjugated to the polypeptides and/or fragments thereof.

The disclosed formulations are stable when stored for prolonged time periods at conventional refrigeration temperatures, e.g., about 2° C. to about 8° C. The formulations exhibit little or no particle agglomeration, no significant decrease in antigen concentration and retain a significant level of immunogenicity and/or antigenicity for at least 6 months or 12 months and preferably for 18 months. The phrase “no significant decrease in antigen concentration” is intended to mean that the composition retains at least 50%, 60%, or 70% of the original antigen concentration, more preferably at least about 80%, 85%, or 90% of the original antigen concentration, more preferably at least about 91%, 92%, 98%, 99% or more of the antigen concentration present when first formulated. Antigen concentration may be measured, for example, by an RP-HPLC, SDS-PAGE or ELISA-based method.

A stable formulation or an immunogenic composition comprising a stable formulation maintains a substantial degree of structural integrity (e.g., maintains a substantial amount of the original antigen concentration, etc.).

Stability may be assessed by measuring for example, the concentration of antigen present (e.g., by RP-HPLC) or by assessing antigen degradation for example by SDS-PAGE analysis. The antigen concentration in the formulation may be compared with that of the formulation as prepared with the same aluminum compound albeit untreated (i.e., not treated with phosphate or carbonate ions). Stability prediction and/or comparison tools include for example, Stability System™ (by ScienTek Software, Inc.), which use Arrhenius Treatment to predict rate constant at storage temperature (2° C.-8° C.). Standard assays for measuring the antigen concentration, and immunogenicity are known in the art and are described in the Examples. Protective efficacy may be assessed by for example evaluating the survival rates of immunized and non-immunized subjects following challenge with a disease causing pathogen or toxin corresponding to the particular antigen present in the formulation.

The immunogenic compositions of the present invention are preferably in liquid form, but they may be lyophilized (as per standard methods) or foam dried (as described in WO2009012601, Antigen-Adjuvant Compositions and Methods). A composition according to one embodiment of the invention is in a liquid form. An immunization dose may be formulated in a volume of between 0.5 and 1.0 ml. Liquid formulations may be in any form suitable for administration including for example, a solution, or suspension. Thus, the compositions can include a liquid medium (e.g., saline or water), which may be buffered.

The pH of the formulation (and composition) is preferably between about 6.4 and about 8.4. More preferably, the pH is about 7.4. An exemplary pH range of the compositions is 5-10, e.g., 5-9, 5-8, 5.5-9, 6-7.5, or 6.5-7. The pH may be maintained by the use of a buffer.

The pharmaceutical formulations of the immunogenic compositions of the present invention may also optionally include one or more excipients (e.g., diluents, thickeners, buffers, preservatives, surface active agents, adjuvants, detergents and/or immunostimulants) which are well known in the art. Suitable excipients will be compatible with the antigen and with the aluminum adjuvant as is known in the art. Examples of diluents include binder, disintegrants, or dispersants such as starch, cellulose derivatives, phenol, polyethylene glycol, propylene glycol or glycerin. Pharmaceutical formulations may also include one or more active ingredients such as antimicrobial agents, antiinflammatory agents and anesthetics. Examples of detergents include a Tween (polysorbate) such as Tween 80. Suitable excipients for inclusion in the composition of the invention are known in the art.

The invention provides compositions including PcpA, PhtX (e.g., PhtD) and/or detoxified pneumolysin proteins and one or more pharmaceutically acceptable excipients that provide beneficial properties to the compositions (e.g., increase the stability of one or more of the proteins of the compositions). The compounds or excipients that can be included in the compositions of the invention include for example, buffers (e.g., glycine, histidine); tonicity agents (e.g., mannitol); carbohydrates, such as sugars or sugar alcohols (e.g., sorbitol, trehalose, or sucrose; 1-30%) or carbohydrate polymers (e.g., dextran); amino acids, oligopeptides or polyamino acids (up to 100 mM); polyhydric alcohols (e.g., glycerol, and concentrations of up to 20%); detergents, lipids, or surfactants (e.g., Tween 20, Tween 80, or pluronics, with concentrations of up to 0.5%); antioxidants; salts (e.g., sodium chloride, potassium chloride, magnesium chloride, or magnesium acetate, up to 150 mM); or combinations thereof.

Examples of excipients that can be used in the compositions of the invention include those that are listed in Table 11, and the examples below. In various examples, the excipients may be those that result in increased thermal stability (e.g., of at least 0.5, e.g., 0.5-5, 1-4, or 2-3) as measured by, e.g., the assays described below (e.g., extrinsic fluorescence of SYPRO Orange).

Exemplary excipients and buffers include sorbitol (e.g., 4-20%, 5-10%), (see Table 11). These excipients can be used in the invention in the concentrations listed in Table 11. Alternatively, the amounts can be varied by, e.g., 0.1-10 fold, as is understood in the art. Other carbohydrates, sugar alcohols, surfactants and amino acids that are known in the art can also be included in the composition of the invention.

The excipients and buffers can be used individually or in combination. The pH of such a composition can be, e.g., 5.5-8.0 or 6.5-7.5, and the composition can be stored at, e.g., 2-8° C., in liquid or lyophilized form. In variations of the composition, the sorbitol can be replaced with sucrose (e.g., 4-20%, or 5-10%), or trehalose (e.g., 4-20%, or 5-10%). Other variations of the compositions are included in the invention and involve use of other components listed herein. Based on the above, an exemplary composition of the invention includes 10% sorbitol, pH 7.4.

In one embodiment, a monovalent PlyD1 composition may include per dose, in the range of 5 to 50 μg of antigen, PTH adjuvant (with about 0.56 mg/mL elemental Aluminum containing 2 mM sodium phosphate buffer at about pH 7.5), in about: 10 mM Tris HCl, and about 150 mM NaCl, at about pH 7.4.

In another embodiment, a monovalent PhtD composition may include per dose, in the range of 5 to 50 μg of antigen, PTH adjuvant (with about 0.56 mg/mL elemental Aluminum containing 2 mM sodium phosphate buffer at about pH 7.5), in about: 10 mM Tris HCl, and about 150 mM NaCl, at about pH 7.4.

In a further embodiment, a monovalent PcpA composition may include per dose, in the range of 5 to 50 μg of antigen, PTH adjuvant (with about 0.56 mg/mL elemental Aluminum containing 2 mM sodium phosphate buffer at about pH 7.5), in about: 10 mM Tris HCl, and about 150 mM NaCl, at about pH 7.4.

In another embodiment, a bivalent formulation composition may include per dose, two proteins (selected from the following: PhtD, PlyD1 or PcpA), each in the range of 5 to 50 μg/dose, PTH adjuvant (with about 0.56 mg/mL elemental Aluminum containing 2 mM sodium phosphate buffer at about pH 7.5), in about: 10 mM Tris HCl, and about 150 mM NaCl, at about pH 7.4.

In yet a further embodiment, a trivalent formulation composition can include per dose, three proteins (PhtD, PlyD1, PcpA), each in the range of 5 to 50 μg/dose, PTH adjuvant (with about 0.56 mg/mL elemental Aluminum containing 2 mM sodium phosphate buffer at about pH 7.5), in about: 10 mM Tris HCl, and about 150 mM NaCl, at about pH 7.4.

In another example, the compositions include sorbitol, or sucrose, which have been shown to provide benefits with respect to stability (see below). The amounts of these components can be, for example, 5-15%, 8-12% or 10% sorbitol or sucrose. A specific example in which these components are present at 10% is described below. In a preferred embodiment the compositions include 10% sorbitol or 10% sucrose.

The invention also includes methods of identifying excipients that can be used to generate compositions including S. pneumoniae proteins (e.g., PcpA, PhtX (e.g., PhtD), detoxified pneumolysin) having improved properties. These methods involve screening assays, such as those described further below, which facilitate the identification of conditions resulting in increased stability of one or more of the protein components of the compositions. These methods include stability assays as described further below. Further, the invention includes the use of other assays for identifying desirable formulations, including solubility, immunogenicity and viscosity assays.

A composition according to one embodiment of the invention may be prepared by (i) treating an aluminum hydroxide adjuvant with phosphate, carbonate, sulfate, carboxylate, diphosphonate or a mixture of two or more of these compounds, and (ii) mixing the treated aluminum hydroxide adjuvant with an immunogenic PcpA polypeptide and/or an immunogenic PhtX polypeptide. In preferred embodiments, the immunogenic PhtX polypeptide is PhtD.

Immunogenic compositions (e.g. vaccines) containing one or more of the S. pneumoniae polypeptides of the present invention may be used to prevent and/or treat S. pneumoniae infections. The prophylactic and therapeutic methods of the invention involve vaccination with one or more of the disclosed immunogenic polypeptides in, for example, carrying out the treatment itself, in preventing subsequent infection, or in the production of antibodies for subsequent use in passive immunization.

The immunogenic compositions of the invention find use in methods of preventing or treating a disease, disorder, condition or symptoms associated with or resulting from a S. pneumoniae infection The terms disease disorder and condition are used interchangeably herein. Specifically the prophylactic and therapeutic methods comprise administration of a therapeutically effective amount of a pharmaceutical composition to a subject. In particular embodiments, methods for preventing or treating S. pneumoniae are provided.

As used herein, preventing a disease or disorder is intended to mean administration of a therapeutically effective amount of a pharmaceutical composition of the invention to a subject in order to protect the subject from the development of the particular disease or disorder associated with S. pneumoniae.

By treating a disease or disorder is intended administration of a therapeutically effective amount of a pharmaceutical composition of the invention to a subject that is afflicted with a disease caused by S. pneumoniae or that has been exposed to S. pneumoniae where the purpose is to cure, heal, alleviate, relieve, alter, remedy, ameliorate, improve, or affect the condition or the symptoms of the disease.

A therapeutically effective amount refers to an amount that provides a therapeutic effect for a given condition and administration regimen. A therapeutically effective amount can be determined by the ordinary skilled medical worker based on patient characteristics (age, weight, gender, condition, complications other diseases etc.). The therapeutically effective amount will be further influenced by the route of administration of the composition.

Also disclosed, is a method of reducing the risk of a pneumococcal disease in a subject comprising administering to the subject an immunogenic composition comprising one or more of the disclosed immunogenic polypeptides. Pneumococcal diseases (i.e., symptomatic infections) include, for example, sinus infection, otitis media, bronchitis, pneumonia, meningitis, hemolytic uremia and bacteremia (septicemia). The risk of any one or more of these infections may be reduced by the methods described herein. Preferred methods include a method of reducing the risk of invasive pneumococcal disease and/or pneumonia in a subject comprising administering to the subject an immunogenic composition comprising an immunogenic PcpA polypeptide and an immunogenic PhtX (e.g., PhtD) polypeptide. In other preferred methods, the composition also includes detoxified pneumolysin (e.g., PlyD1).

The present disclosure also provides methods of eliciting an immune response in a mammal by administering the immunogenic compositions, or formulations thereof, to subjects. This may be achieved by the administration of a pharmaceutically acceptable formulation of the compositions to the subject to effect exposure of the immunogenic polypeptide and/or adjuvant to the immune system of the subject. The administrations may occur once or may occur multiple times. In one example, the one or more administrations may occur as part of a so-called “prime-boost” protocol. Other administration systems may include time-release, delayed release or sustained release delivery systems.

Immunogenic compositions may be presented in a kit form comprising the immunogenic composition and an adjuvant or a reconstitution solution comprising one or more pharmaceutically acceptable diluents to facilitate reconstitution of the composition for administration to a mammal using conventional or other devices. Such a kit would optionally include the device for administration of the liquid form of the composition (e.g. hypodermic syringe, microneedle array) and/or instructions for use.

The compositions and vaccines disclosed herein may also be incorporated into various delivery systems. In one example, the compositions may be applied to a “microneedle array” or “microneedle patch” delivery system for administration. These microneedle arrays or patches generally comprise a plurality of needle-like projections attached to a backing material and coated with a dried form of a vaccine. When applied to the skin of a mammal, the needle-like projections pierce the skin and achieve delivery of the vaccine, effecting immunization of the subject mammal.

### DEFINITIONS

The term “antigen” as used herein refers to a substance that is capable of initiating and mediating the formation of a corresponding immune body (antibody) when introduced into a mammal or can be bound by a major histocompatibility complex (MHC) and presented to a T-cell. An antigen may possess multiple antigenic determinants such that the exposure of the mammal to an antigen may produce a plurality of corresponding antibodies with differing specificities. Antigens may include, but are not limited to proteins, peptides, polypeptides, nucleic acids and fragments, variants and combinations thereof.

The term “immunogen” is a substance that is able to induce an adaptive immune response.

The terms peptides, proteins and polypeptides are used interchangeably herein.

An “isolated” polypeptide is one that has been removed from its natural environment. For instance, an isolated polypeptide is a polypeptide that has been removed from the cytoplasm or from the membrane of a cell, and many of the polypeptides, nucleic acids, and other cellular material of its natural environment are no longer present. An “isolatable” polypeptide is a polypeptide that could be isolated from a particular source. A “purified” polypeptide is one that is at least 60% free, preferably at least 75% free, and most preferably at least 90% free from other components with which they are naturally associated. Polypeptides that are produced outside the organism in which they naturally occur, e.g. through chemical or recombinant means, are considered to be isolated and purified by definition, since they were never present in a natural environment.

As used herein, a “fragment” of a polypeptide preferably has at least about 40 residues, or 60 residues, and preferably at least about 100 residues in length. Fragments of S. pneumoniae polypeptides can be generated by methods known to those skilled in the art.

The term “antibody” or “antibodies” includes whole or fragmented antibodies in unpurified or partially purified form (i.e., hybridoma supernatant, ascites, polyclonal antisera) or in purified form. A “purified” antibody is one that is separated from at least about 50% of the proteins with which it is initially found (i.e., as part of a hybridoma supernatant or ascites preparation).

As used in the specification and the appended claims, the singular forms “a”, “an”, and “the” include plural referents unless the context clearly dictates otherwise. Thus, for example, reference to a fragment may include mixtures of fragments and reference to a pharmaceutical carrier or adjuvant may include mixtures of two or more such carriers or adjuvants.

As used herein, a subject or a host is meant to be an individual.

Optional or optionally means that the subsequently described event or circumstance can or cannot occur, and that the description includes instances where the event or circumstance occurs and instances where it does not. For example, the phrase, “optionally the composition can comprise a combination” means that the composition may comprise a combination of different molecules or may not include a combination such that the description includes both the combination and the absence of the combination (i.e., individual members of the combination).

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint.

When the terms prevent, preventing, and prevention are used herein in connection with a given treatment for a given condition (e.g., preventing S. pneumoniae infection), it is meant to convey that the treated subject either does not develop a clinically observable level of the condition at all, or develops it more slowly and/or to a lesser degree than he/she would have absent the treatment. These terms are not limited solely to a situation in which the subject experiences no aspect of the condition whatsoever. For example, a treatment will be said to have prevented the condition if it is given during exposure of a patient to a stimulus that would have been expected to produce a given manifestation of the condition, and results in the subject's experiencing fewer and/or milder symptoms of the condition than otherwise expected. A treatment can “prevent” infection by resulting in the subject's displaying only mild overt symptoms of the infection; it does not imply that there must have been no penetration of any cell by the infecting microorganism.

Similarly, reduce, reducing, and reduction as used herein in connection with the risk of infection with a given treatment (e.g., reducing the risk of a S. pneumoniae infection) refers to a subject developing an infection more slowly or to a lesser degree as compared to a control or basal level of developing an infection in the absence of a treatment (e.g., administration of an immunogenic polypeptide). A reduction in the risk of infection may result in the subject displaying only mild overt symptoms of the infection or delayed symptoms of infection; it does not imply that there must have been no penetration of any cell by the infecting microorganism.

All references cited within this disclosure are hereby incorporated by reference in their entirety.

### EXAMPLES

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations.

Methods of molecular genetics, protein biochemistry, immunology and fermentation technology used, but not explicitly described in this disclosure and these Examples, are amply reported in the scientific literatures and are well within the ability of those skilled in the art.

### Example 1A

### Recombinant PcpA and PhtD Polypeptides

This Example describes the preparation of the PcpA protein and PhtD protein recombinantly. In brief, two recombinantly-derived protein antigens from Streptococcus pneumoniae (strain 14453 (a mouse-virulent capsule serotype 6B strain), deposited on Jun. 27, 1997 as ATCC 55987), PhtD (WO2009/012588) and PcpA (WO 2008/022302) were recombinantly expressed in E. coli, isolated and purified by serial column chromatography following conventional purification protocols.

The phtD gene (but excluding its native signal peptide) was PCR amplified from the S. pneumoniae 14453 genome, using the AccuPrime High Fidelity polymerase (Invitrogen) and primers Spn0211 and Spn0213. Spn0211 and Spn0213 introduced NocI and XhoI restriction sites into the 5′ and 3′ ends, respectively (see Table 2). The PCR product was purified using a QIAquick PCR purification kit (Qiagen) and run on an agarose gene to confirm the size. The PCT product and the pET28a(+) vector (Novagen) were both digested with NcoI and XhoI and subsequently purified from an agarose gel using the QIAEX gel extraction kit (Qiagen). The digested vector and gene were ligated together using T4 DNA ligase (Invitrogen). The ligation mixture was transformed into chemically competent E. coli DH5α and positive clones were selected by plating on Luria agar containing 50 μg/ml kanamycin. DNA from plasmid clone pBAC27 was isolated and was confirmed by sequencing to be correct.

The plasmid (pBAC27) was then introduced into E. coli BL21 (DE3) cells by electroporation. Transformed strains were grown at approximately 37° C. and protein expression was induced by the addition of 1 mM IPTG. Expression of gene product was verified by the presence of an induced protein band of the correct size (i.e., approximately 91.9 kDa) by SDS-PAGE analysis.

TABLE 2

| Primer Name/Number | Sequence 5′ → 3′ |
|---|---|
| Spn0211 | CTAGCCATGGGATCCTATGAACTTGGTCGTC |
| | ACCAAG |
| Spn0213 | AGTCCTCGAGCTACTGTATAGGAGCCGGTTG |

The predicted amino acid sequence of the polypeptide of pBAC27 is as follows:

(SEQ ID No: 5)
MGSYELGRHQAGQVKKESNRVSYIDGDQAGQKAENLTPDEVSKREGINAEQIVIKITDQGYVTSHGDHYHYY
NGKVPYDAIISEELLMKDPNYQLKDSDIVNEIKGGYVIKVDGKYYVYLKDAAHADNIRTKEEIKRQKQEHSH
NHNSRADNAVAAARAQGRYTTDDGYIFNASDIIEDTGDAYIVPHGDHYHYIPKNELSASELAAAEAYWNGKQ
GSRPSSSSSYNANPVQPRLSENHNLTVTPTYHQNQGENISSLLRELYAKPLSERHVESDGLIFDPAQITSRT
ARGVAVPHGNHYHFIPYEQMSELEKRIARIIPLRYRSNHWVPDSRPEQPSPQSTPEPSPSLQPAPNPQPAPS
NPIDEKLVKEAVRKVGDGYVFEENGVSRYIPAKDLSAETAAGIDSKLAKQESLSHKLGAKKTDLPSSDREFY
NKAYDLLARIHQDLLDNKGRQVDFEVLDNLLERLKDVSSDKVKLVDDILAFLAPIRHPERLGKPNAQITYTD
DEIQVAKLAGKYTTEDGYIFDPRDITSDEGDAYVTPHMTHSHWIKKDSLSEAERAAAQAYAKEKGLIPPSTD
HQDSGNTEAKGAEAIYNRVKAAKKVPLDRMPYNLQYTVEVKNGSLIIPHYDHYHNIKFEWFDEGLYEAPKGY
SLEDLLATVKYYVEHPNERPHSDNGFGNASDHVRKNKADQDSKPDEDKEHDEVSEPTHPESDEKENHAGLNP
SADNLYKPSTDTEETEEEAEDTTDEAEIPQVENSVINAKIADAEALLEKVTDPSIRQNAMETLTGLKSSLLL
GTKDNNTISAEVDSLLALLKESQPAPIQ

The pcpA gene (but excluding the signal sequence and the choline-binding domains) was PCR amplified from the S. pneumoniae 14453 genome using Accuprime Taq DNA polymerase (Invitrogen) and PCR primers (see Table 3) that incorporated restriction endonuclease sites designed for simplified cloning. Plasmid DNA of pET-30a(+) (Novagen) was purified as a low-copy plasmid and prepared for use as the cloning vector by digesting with NdeI and XhoI, followed by gel purification. The resulting 1335 base pair fragment was pcpA (without signal sequence and choline-binding domains) flanked by XhoI (3′-end) and NdeI (5′ end) restriction sites. The amplified fragment was cleaned, digested with NdeI and XhoI and then gel purified and ligated into the pET-30a(+) vector. The insert was verified by sequencing and the new plasmid was designated pJMS87.

TABLE 3
(Primers)

| Primer Name | Sequence 5′ → 3′ |
|---|---|
| UAB 3 | TAGCCTCGAGTTAACCTTTGTCTTTAACCCAACC |
| | AACTACTCCCTGATTAG |
| UAB-tagless 5 | CTAATGAACCACATATGGCAGATACTCCTAGTTC |
| | GGAAGTAATC |

The predicted amino acid sequence of the polypeptide of pJMS87 is as follows:

(SEQ ID No: 7)
MADTPSSEVIKETKVGSTIQQNNIKYKVLTVEGNIGTVQVGNGVTPVEFEAGQDGKPFTIPTKITVGDKVFT
VTEVASQAFSYYPDETGRIVYYPSSITIPSSIKKIQKKGFHGSKAKTIIFDKGSQLEKIEDRAFDFSELEEI
ELPASLEYIGTSAFSFSQKLKKLTFSSSSKLELISHEAFANLSNLEKLTLPKSVKTLGSNLFRLTTSLKHVD
VEEGNESFASVDGVLFSKDKTQLIYYPSQKNDESYKTPKETKELASYSFNKNSYLKKLELNEGLEKIGTFAF
ADAIKLEEISLPNSLETIERLAFYGNLELKELILPDNVKNFGKHVMNGLPKLKSLTIGNNINSLPSFFLSGV
LDSLKEIHIKNKSTEFSVKKDTFAIPETVKFYVTSEHIKDVLKSNLSTSNDIIVEKVDNIKQETDVAKPKKN
SNQGVVGWVKDKG

Chemically competent E. coli BL21 (DE3) cells were transformed with plasmid pJMS87 DNA. Expression of gene product was verified by the presence of an induced protein band of the correct size (i.e., approximately 49.4 kDa) by SDS-PAGE analysis.

As the cloned PcpA polypeptide lacks the signal sequence and choline-binding domains, its amino acid sequence correlates with amino acids 27 to 470 of the full length PcpA protein. This region is extremely conserved among all surveyed strains with only 8 variable positions. The most diverged pair of sequences shares 98.7% identity.

The predicted isoelectric points by Vector NTi for the recombinant PcpA protein and the recombinant PhtD protein were 7.19 and 5.16, respectively.

The pcpA gene and phtD gene were each detected in the following serotypes: 1, 2, 3, 4, 5, 6A, 6B, 6C, 7, 7F, 9N, 9V, 11A/B, 11A/D/F, 12F/B, 14, 15B, 15B/C, 16, 18C, 19A, 19F, 22, 23, 23B, 23F, 33F, 34, 35B. A number of these serotypes are not covered by the currently marketed pneumococcal conjugate vaccine PCV7.

The recombinant protein products were expressed, isolated and purified using standard methods.

Adjuvanted monovalent compositions of either recombinant protein were prepared by formulating isolated purified protein with adjuvant (e.g., Aluminum hydroxide adjuvant (e.g. Alhydrogel 85 2%) or AlPO₄) in Tris buffered saline (pH 7.4) using standard methods. Formulated materials were transferred to glass vials and stored at 2° C. to 8° C. Adjuvanted bivalent compositions of both PhtD and PcpA were prepared by aliquoting the desired concentration of each adjuvanted monovalent formulation into a vessel and mixing on a nutator for approximately 0.5 hours at room temperature. Desired formulation volumes were then aliquoted into sterile 3 mL glass vials with rubber stopper closure and aluminum cap. Alternatively, bivalent compositions were prepared by mixing the desired concentration of each isolated purified protein together and then formulating mixture with adjuvant in Tris buffered saline (pH 7.4).

### Example 1B

This Example describes the preparation of a surface modified adjuvant and formulations with this adjuvant. A surface modified adjuvant was prepared by treating aluminum hydroxide adjuvant (Alhydrogel™, Brenntag) with phosphate. The aluminum hydroxide adjuvant used was a wet gel suspension which according to the manufacturer tolerates re-autoclavation but is destroyed if frozen. According to the manufacturer, when the pH is maintained at 5-7, the adjuvant has a positive charge and can adsorb negatively charged antigens (e.g., proteins with acidic isoelectric points when kept at neutral pH).

a) Phosphate treatment of AlO(OH)—An aqueous suspension of AlO(OH) (approximately 20 mg/mL) was mixed with a stock solution of phosphate buffer (approximately 400 mol/L) and diluted with 10 mM Tris-HCL buffer (Sigma Aldrich) at about pH 7.4 to prepare a phosphate-treated AlO(OH) suspension (herein referred to as “PTH”) having approximately 13 mg/mL AlOOH/200 mM PO4. This suspension was then mixed for approximately 30 minutes to 24 hr at room temperature.
b) Antigen adsorption—Recombinantly-derived PcpA and PhtD antigens (expressed, isolated and purified as described in Example 1A) were individually adsorbed to the phosphate-treated AlO(OH).

A mixture was prepared containing about 0.2-0.4 mg/mL of purified antigen (i.e., rPcpA or rPhtD) each antigen and 0.56 mg elemental aluminum/ml/PO4 mM of the PTH suspension. Alternatively, mixtures were prepared containing purified antigen with aluminum hydroxide adjuvant (as Alhydrogel® 85 2%) or AlPO4 in Tris buffered saline (pH 7.4) using standard methods. The mixtures wereas mixed in an orbital shaker for about 30 minutes to 24 hours at room temperature to facilitate the association of antigen and adjuvant. Similar adsorptions were prepared a number of times and the typical pre-adsorbed composition was: protein (PhtD or PcpA): 0.2-0.4 mg/ml, phosphate: 2 to 20 80 mM (preferably, 2 to 20 mM) and AlO(OH): 1.25 mg/ml (0.56 mg of elemental Al/ml). Prepared antigen adsorbed samples were stored at about 2° C.-8° C. until used. Alternatively, antigens were adjuvanted together (to prepare bivalent formulations) by using a stock solution of phosphate treated aluminum hydroxide adjuvant.

c) Preparation of a bivalent formulation—The intermediate bulk lots (monovalent formulations) of PhtD adsorbed to PTH and PcpA adsorbed to PTH were blended and mixed together for about 30 minutes at room temperature in an orbital shaker to prepare a bivalent formulation. The typical pre-adsorbed formulation composition was: 0.05 mg/ml of each protein (rPhtD, rPcpA); phosphate: 2 to 20 mM and 1.25 mg/mL AlO(OH) (0.56 mg of elemental Al/ml).

### Example 2

### Assessment of Antigenic Interference and Humoral Response with Bivalent Compositions Formulated with Varying Doses of PcpA and PhtD

This Example describes the analysis of the immunogenicity of a multi-component composition in animals. Formulations were prepared (as described in Example 1) using purified PhtD and PcpA proteins, aluminum hydroxide adjuvant (Alhydrogel® 85 2%, 25.52 mg/mL), Tris buffered saline (10 mM Tris-HCl pH 7.4/150 mM NaCl). The formulations were mixed on a Nutator for approximately 30 minutes and dispensed into glass vials.

Groups of 10 female mice Balb/c K-72 mice (Charles River), 6 to 8 weeks of age, were immunized subcutaneously (SC) three times at 3 week intervals with the applicable formulation:

A. (5 μg/mL of PcpA+1.3 mg/mL AlOOH) in Tris buffered saline pH=7.4
B. (12.5 μg/mL of PcpA+1.3 mg/mL AlOOH) in Tris buffered saline pH=7.4
C. (25 μg/mL of PcpA+1.3 mg/mL AlOOH) in Tris buffered saline pH=7.4
D. (5 μg/mL of PhtD+1.3 mg/mL AlOOH) in Tris buffered saline pH=7.4
E. (12.5 μg/mL of PhtD+1.3 mg/mL AlOOH) in Tris buffered saline pH=7.4
F. (25 μg/mL of PhtD+1.3 mg/mL AlOOH) in Tris buffered saline pH=7.4
G. (5 μg/mL of PcpA+5 μg/mL PhtD+1.3 mg/mL AlOOH) in Tris buffered saline pH=7.4
H. (5 μg/mL of PcpA+12.5 μg/mL PhtD+1.3 mg/mL AlOOH) in Tris buffered saline pH=7.4
I. (5 μg/mL of PcpA+25 μg/mL PhtD+1.3 mg/mL AlOOH) in Tris buffered saline pH=7.4
J. (12.5 μg/mL of PcpA+5 μg/mL PhtD+1.3 mg/mL AlOOH) in Tris buffered saline pH=7.4
K. (12.5 μg/mL of PcpA+12.5 μg/mL PhtD+1.3 mg/mL AlOOH) in Tris buffered saline pH=7.4
L. (12.5 μg/mL of PcpA+25 μg/mL PhtD+1.3 mg/mL AlOOH) in Tris buffered saline pH=7.4
M. (25 μg/mL of PcpA+5 μg/mL PhtD+1.3 mg/mL AlOOH) in Tris buffered saline pH=7.4
N. (25 μg/mL of PcpA+12.5 μg/mL PhtD+1.3 mg/mL AlOOH) in Tris buffered saline pH=7.4
O. (25 μg/mL of PcpA+25 μg/mL PhtD+1.3 mg/mL AlOOH) in Tris buffered saline pH=7.4

Sample bleeds were taken from all animals 2 days prior to first immunization and following the first, second and third immunizations. Blood samples from individual mice were centrifuged at 9,000 rpm for 5 minutes and the recovered sera were stored at −20° C.

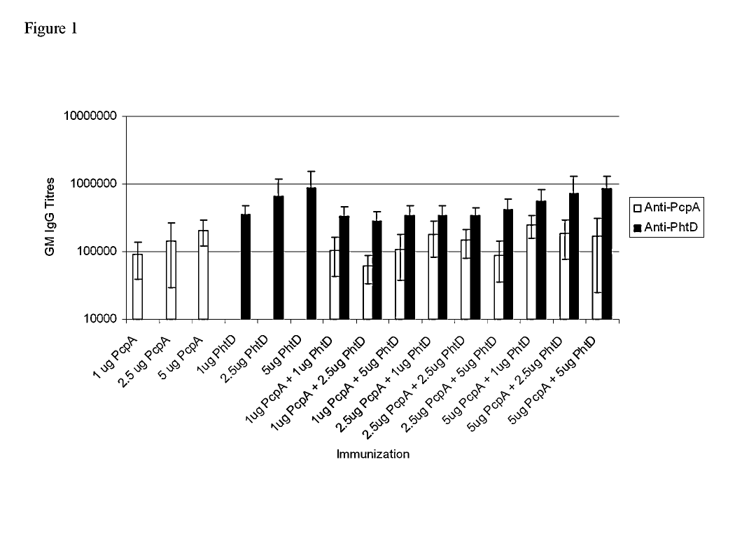

Total antigen-specific IgG titres were measured in pooled prebleeds and in sera collected following the first, second and third immunizations by endpoint dilution ELISA and geometric mean titres for each group are shown in FIG. 1. The antibody titers in the prebleeds were below the limit of detection (<100), while the final bleed titers for both PhtD and PcpA monovalent formulations were high for both antigens in all groups consistent with those observed from previous studies. PhtD and PcpA-specific antibody ELISA titers are summarized in Table 4.

TABLE 4
PcpA and PhtD-specific ELISA Titers for Groups of Mice Immunized
with Monovalent or Bivalent Formulation

| | | ELISA Titers | |
|---|---|---|---|
| Formulation | Bleed* | PcpA | PhtD |
| 1μg PcpA | Pre-immunization | <100 | <100 |
| | Final bleed | 77605 | 100 |
| 2.5 μg PcpA | Pre-immunization | <100 | <100 |
| | Final bleed | 110598 | 100 |
| 5 μg PcpA | Pre-immunization | <100 | <100 |
| | Final bleed | 191085 | 100 |
| 1 μg PhtD | Pre-immunization | <100 | <100 |
| | Final bleed | <100 | 332699 |
| 2.5 μg PhtD | Pre-immunization | <100 | <100 |
| | Final bleed | <100 | 540470 |
| 5 μg PhtD | Pre-immunization | <100 | <100 |
| | Final bleed | <100 | 620838 |
| 1 μg PcpA + 1 μg PhtD | Pre-immunization | <100 | <100 |
| | Final bleed | 89144 | 289631 |
| 1 μg PcpA + 2.5 ug PhtD | Pre-immunization | <100 | <100 |
| | Final bleed | 55834 | 265593 |
| 1 μg PcpA + 5 ug PhtD | Pre-immunization | <100 | <100 |
| | Final bleed | 89144 | 310419 |
| 2.5 μg PcpA + 1 μg PhtD | Pre-immunization | <100 | <100 |
| | Final bleed | 162550 | 301002 |
| 2.5 μg PcpA + 2.5 μg PhtD | Pre-immunization | <100 | <100 |
| | Final bleed | 126069 | 332699 |
| 2.5 μg PcpA + 5 μg PhtD | Pre-immunization | <100 | <100 |
| | Final bleed | 75250 | 378460 |
| 5 μg PcpA + 1 μg PhtD | Pre-immunization | <100 | <100 |
| | Final bleed | 238905 | 477810 |
| 5 μg PcpA + 2.5 μg PhtD | Pre-immunization | <100 | <100 |
| | Final bleed | 157922 | 579262 |
| 5 μg PcpA + 5 μg PhtD | Pre-immunization | <100 | <100 |
| | Final bleed | 117627 | 764341 |

*Final bleed anti-PcpA and PhtD titers were determined from individual mice and are represented as the geometrical mean.

Statistical analysis of the ELISA data investigated the effect of PcpA concentration on the anti-PhtD responses that were elicited (following the third immunization) by the bivalent formulations in comparison to the anti-PhtD responses that were elicited by the monovalent PhtD formulations. Similarly, the effect of PhtD concentration on the anti-PcpA responses that were elicited (following the third immunization) by the bivalent formulations in comparison to the anti-PcpA responses that were elicited by the monovalent PcpA formulations was also assessed. With respect to the anti-PcpA IgG titers, no statistically significant differences were observed when comparing the responses elicited by the monovalent PcpA formulations to those elicited by the bivalent formulations (9/9 groups). Therefore, no statistically significant interaction, either positive or negative, with PhtD was observed at any dose examined. In regards to the anti-PhtD titres, in most comparisons between the anti-PhtD titres (i.e., responses) elicited by the bivalent formulations and those elicited by the corresponding monovalent PhtD formulations, no statistically significant inhibition was noted (7/9 groups). Two exceptions were observed, each showing a two-fold decrease in anti-PhtD titers: (i) the bivalent formulation containing PcpA at 1 μg/dose and PhtD at 2.5 μg/dose in comparison to the monovalent formulation of PhtD at 2.5 μg/dose (p=0.034); and (ii) the bivalent formulation containing PcpA at 1 μg/dose and PhtD at 5.0 μg/dose in comparison to the monovalent formulation of PhtD at 5.0 μg/dose (p=0.027). Statistical significance was not observed for the 1 μg dose of PhtD, nor with the higher doses of PcpA (i.e., 2.5 μg and 5 μg). However, this two fold decrease is within the range of variability of the model and thus does not reflect significant levels of interference.

The optimum concentration of each antigen (PcpA, PhtD) in a bivalent composition as determined by statistical analysis was 25 μg/mL (i.e., 5 μg/dose). Monovalent compositions with this concentration of antigen (i.e., 25 μg/mL of PcpA or PhtD) also elicited the highest antigen specific IgG titres.

### Example 3

### Immunogenicity Study in Rats Following 3 Intramuscular Injections of the Bivalent Vaccine

This Example describes the analysis of the safety and immunogenicity of a multi-component vaccine in another animal species (i.e., rat).

Four groups of (20 per sex) Wistar Crl:WI (Han) rats were given 3 IM injections of either control, bivalent vaccine composition with or without adjuvant or adjuvanted monovalent PcpA vaccine composition at three weekly intervals on Days 0, 21 and 42 (see study design in Table 5 below). Animals were necropsied on Days 2 or 15 after the last administration. Compositions were prepared as described in Example 1. The adjuvant used to prepare adjuvanted compositions was aluminum hydroxide (Alhydrogel®, Brenntag). See Table 5 for an outline summary of the study design.

TABLE 5
(Study Design)

| | Dose Level | | | |
|---|---|---|---|---|
| | (μg/dose/ | Dose Level | Number of Animals | |
| Group | administration) | (μl/animal) | Male | Female |
| Control (Tris | 0 | 2 × 250 | 20 | 20 |
| Buffer Saline) | | | | |
| PhtD/PcpA with | 50 | 2 × 250 | 20 | 20 |
| Adjuvant | | | | |
| PhtD/PcpA | 50 | 2 × 250 | 20 | 20 |
| without | | | | |
| Adjuvant | | | | |
| PcpA with | 50 | 2 × 250 | 20 | 20 |
| Adjuvant | | | | |

Morbidity/mortality checks were performed at least twice daily and clinical examinations were performed daily. There were no premature deaths, adverse clinical signs, effects on body weight, food consumption, clinical chemistry or ophthalmology that were considered treatment related.

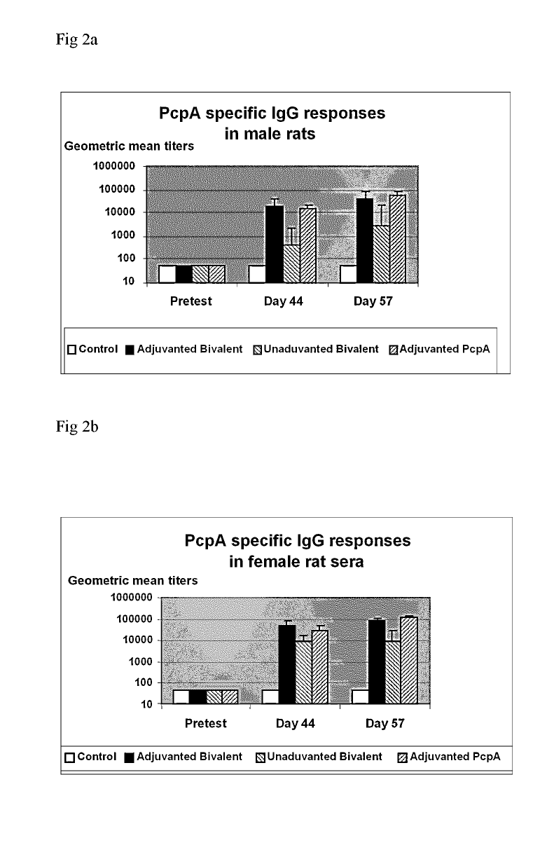

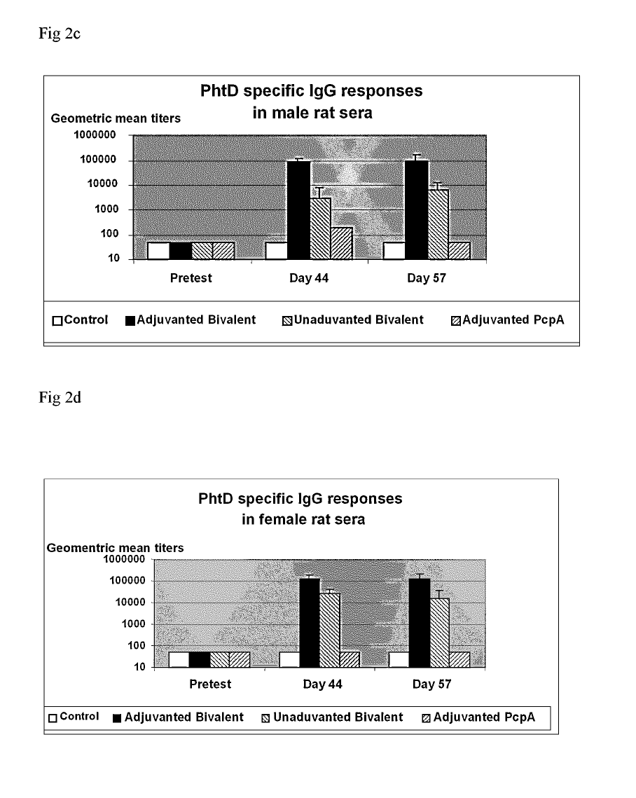

Sera were analyzed for PhtD and PcpA specific IgG antibody titers by ELISA. The results are set out in FIGS. 2 a to d. All treated animals showed robust anti-PcpA and anti-PhtD responses, although the responses in the unadjuvanted group were more variable. Adjuvanted monovalent PcpA vaccine elicited an immune response that was equivalent to the adjuvanted bivalent vaccine, indicating the absence of immunological interference by PhtD in the bivalent formulation.

The bivalent and PcpA monovalent vaccine compositions each induced an immune response in all animals. According to the results here, the bivalent and PcpA monovalent vaccine compositions are immunogenic in rats. Adjuvanted compositions were more immunogenic than unadjuvanted compositions.

### Example 4

### Assessing Immunogenicity of Bivalent Composition Formulated with Different Aluminum-Based Adjuvants

This Example describes the analysis of the immunogenicity of a multi-component composition formulated with different aluminum-based adjuvants.

In one study, recombinant PhtD and PcpA (prepared and purified as described in Example 1) were formulated with either fresh aluminum hydroxide adjuvant (Alhydrogel®), aged aluminum hydroxide adjuvant (Alhydrogel®, Brenntag), which had been incubated at 2-8° C. for approximately 6 months, aluminum hydroxide adjuvant (Alhydrogel®, Brenntag) treated with various concentrations of phosphate PO₄ (2 mM, 10 mM and 20 mM) or AlPO₄ (Adjuphos®, Brenntag). Formulations were prepared as described in Example 1. Groups of 5 (or 4) female Balb/c mice (Charles River), 6-8 weeks of age upon arrival, were immunized intramuscularly (IM) three times at 3 week intervals with the applicable formulation. The specific formulations administered to each group is set out in Table 6.

The PhtD and PcpA-specific antibody ELISA titers following the final bleed are summarized in Table 6. Mice immunized with PcpA and/or PhtD proteins generated antigen-specific antibody responses after immunization. No significant differences in anti-PhtD and anti-PcpA titres were seen in animals immunized with bivalent formulations with either fresh or aged AlOOH or pre-treated with phosphate (at any of the three concentrations used). Immunization with the bivalent composition formulated with AlPO₄ (which is less immunogenic than AlOOH) gave rise to significantly lower anti-PhtD IgG titres when compared to formulations containing AlOOH or PO₄-containing AlOOH adjuvants. These results were confirmed in other studies that compared bivalent compositions formulated with aluminum hydroxide adjuvant and AlPO4 adjuvants.

In total, four studies were completed using both recombinant PcpA and PhtD as immunogens formulated with aluminum-based adjuvants (aluminum hydroxide adjuvant, aluminum hydroxide adjuvant treated with various concentrations of PO₄, AlPO₄). Both antigens were given at various doses ranging from 1-5 μg/dose. Specific PcpA and PhtD antibody titers were determined in pooled prebleeds and in sera collected following three IM or SC immunizations. The antibody titers in the prebleeds were below the limit of detection (<100), while the final bleed titers were ranged between 124827 to 204800 for anti-PcpA and 36204 to 97454 for anti-PhtD.

In sum, according the results here, compositions formulated with any of the adjuvants tested were immunogenic. Immunization with recombinant PhtD and PcpA proteins formulated with aluminum hydroxide adjuvants (i.e. aluminum hydroxide adjuvant and aluminum hydroxide adjuvant treated with phosphate) generated significantly higher antigen-specific antibody responses (IgG tiers) to both PcpA and PhtD in comparison to immunizations with AlPO₄ formulations.

TABLE 6
PcpA and PhtD-specific ELISA Titers for Groups of Mice Immunized
with Placebo or Bivalent Vaccine Formulation

| | | ELISA Titers | |
|---|---|---|---|
| Group | Bleed* | PcpA | PhtD |
| 5 μg PcpA + PhtD + AlOOH | Pre-immunization | <100 | <100 |
| | Final bleed | 152166 | 88266 |
| 5 μg PcpA + PhtD + AlOOH | Pre-immunization | <100 | <100 |
| with 2 mM PO₄ | Final bleed | 204800 | 88266 |
| 5 μg PcpA + PhtD + AlOOH | Pre-immunization | <100 | <100 |
| with 10 mM PO₄ | Final bleed | 204800 | 64508 |
| 5 μg PcpA + PhtD + AlOOH | Pre-immunization | <100 | <100 |
| with 20 mM PO₄ | Final bleed | 176532 | 68910 |
| 10 μg PcpA + PhtD + fresh | Pre-immunization | <100 | <100 |
| AlOOH | Final bleed | 176532 | 97454 |
| 10 μg PcpA + PhtD + aged | Pre-immunization | <100 | <100 |
| AlOOH | Final bleed | 168005 | 88266 |
| 5 μg PcpA + PhtD + AlPO4 | Pre-immunization | <100 | <100 |
| | Final bleed | 124827 | 36204 |

*Final bleed anti-PcpA and anti-PhtD titers were determined from individual mice and are represented as the geometrical mean.

### Example 5

### Survival Following Challenge with S. pneumoniae Strains 14453, MD or 941192

This Example describes the protective ability of a multi-component vaccine against fatal pneumococcal challenge in the mouse intranasal challenge model.

A bivalent formulation of recombinant PhtD and PcpA was evaluated using an intranasal (IN) challenge model. In this model, groups of female CBA/j mice (N=15 per group) were immunized intramuscularly (IM) with a bivalent composition containing a 5 μg/dose of each of purified recombinant PhtD and PcpA proteins, formulated in TBS with adjuvant (AlOOH treated with 2 mM PO₄ (65 μg/dose)). The injection volume was 50 μL per dose. As a negative control, a PBS placebo-containing aluminum adjuvant was injected. Animals were immunized IM at 0, 3, and 6 weeks following initiation of the study. At 9 weeks, animals were administered a lethal dose (approximately 106 CFU) intranasally of an S. pneumoniae strain MD, strain 14453 or 941192 in PBS suspension (40 μL challenge volume per mouse). Sample bleeds were taken from all animals 4 days prior to the first injection (pre-immunization at 0 weeks) and 4 days prior to the challenge. Sera were analyzed for total PhtD and PcpA-specific IgG response by means of an antibody ELISA assay.

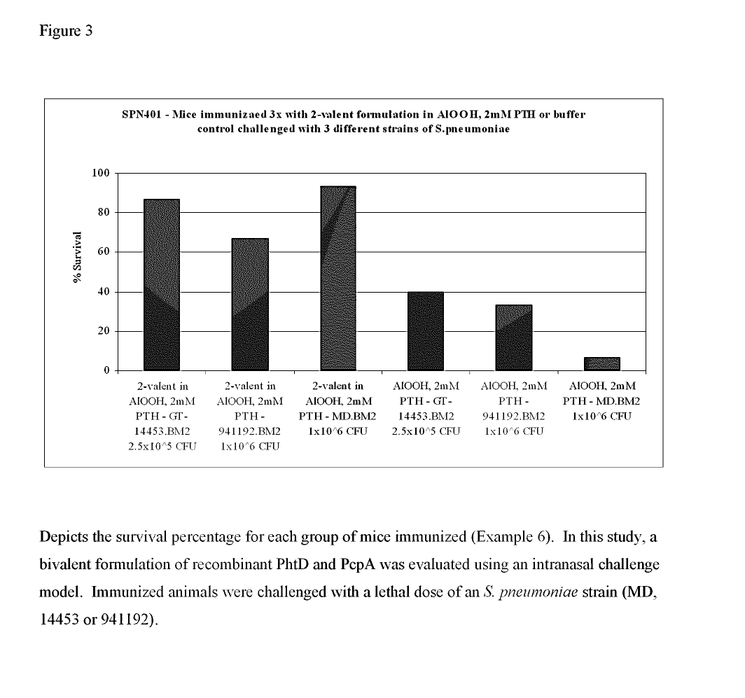

Following the challenge, mice were monitored daily for mortality. All surviving mice were euthanized 11 days post-challenge. Protection was determined using Fisher's one-sided Exact test by comparing survival in the immunized group(s) to the placebo control (p values <0.05 were considered significant). The results of the study (noted in % survival) are set out in FIG. 3 and Table 7 below.

TABLE 7
Survival Results of Mice Immunized with Bivalent Vaccine or Placebo

| | Bivalent Survival in % | | Placebo Survival in % | |
|---|---|---|---|---|
| Day | Strain 14453 | Strain MD | Strain 14453 | Strain MD |
| 0 | 100 | 100 | 100 | 100 |
| 1 | 100 | 100 | 100 | 100 |
| 2 | 100 | 93.3 | 73.3 | 20 |
| 3 | 100 | 93.3 | 40 | 6.7 |
| 4 | 86.7 | 93.3 | 40 | 6.7 |
| 5 | 86.7 | 93.3 | 40 | 6.7 |
| 6 | 86.7 | 93.3 | 40 | 6.7 |
| 7 | 86.7 | 93.3 | 40 | 6.7 |
| 8 | 86.7 | 93.3 | 40 | 6.7 |
| 9 | 86.7 | 93.3 | 40 | 6.7 |
| 10 | 86.7 | 93.3 | 40 | 6.7 |
| 11 | 86.7 | 93.3 | 40 | 6.7 |
| p-value* | 0.01 | 0.000 | | |

*p-value calculated using the Fisher exact test versus placebo group; difference from placebo group 11 days post-challenge

Immunization with combined recombinant PhtD and PcpA proteins generated protection against fatal IN challenge with three different strains of S. pneumoniae in the IN challenge model. The protection noted in groups that had been challenged with either the 14453 strain or the MD strain was statistically significant. The group challenged with the 941192 strain also had a high % survival, but the protection was not considered statistically significant in light of the percentage of survival noted in the negative control group (immunized with adjuvant alone).

### Example 6

### Humoral Response and Survival Following Challenge Using Different Routes of Administration (Subcutaneous or Intramuscular)

This Example describes the protective ability of a multi-component vaccine against fatal pneumococcal challenge in the mouse intranasal challenge model.

Bivalent compositions of PhtD and PcpA were prepared (using two different lots of each of rPhtD and rPcpA) and were formulated with an aluminum hydroxide adjuvant (AlOOH) that was pre-treated with 2 mM of phosphate (according to process described in a patent application filed concurrently with this application). The prepared formulations were evaluated in the mouse active immunization intranasal challenge model (based on a model described in Zhang Y. A. et. al., Infect. Immunol. 69:3827-3836). More specifically, 16 groups of 6 female CBA/j mice (Charles River), 6-8 weeks of age upon arrival, were immunized intramuscularly or subcutaneously three times at 3 week intervals with the applicable formulation:

A. PcpA Lot A, PhtD Lot C, Unadjuvanted, s.c. (25 μg/ml/protein)
B. PcpA Lot B, PhtD Lot C, Unadjuvanted, s.c. (25 μg/ml/protein)
C. PcpA Lot A, PhtD Lot D, Unadjuvanted, s.c. (25 μg/ml/protein)
D. PcpA Lot B, PhtD Lot D, Unadjuvanted, s.c. (25 μg/ml/protein)
E. PcpA Lot A, PhtD Lot C+2 mM phosphate treated AlOOH, s.c. (25 μg/ml/protein)
F. PcpA Lot B, PhtD Lot C+2 mM phosphate treated AlOOH, s.c. (25 μg/ml/protein)
G. PcpA Lot A, PhtD Lot D+2 mM phosphate treated AlOOH, s.c. (25 μg/ml/protein)
H. PcpA Lot B, PhtD Lot D+2 mM phosphate treated AlOOH, s.c. (25 μg/ml/protein)
I. PcpA Lot A, PhtD Lot C Unadjuvanted, i.m. (100 μg/ml/protein)
J. PcpA Lot B, PhtD Lot C Unadjuvanted, i.m. (100 μg/ml/protein)
K. PcpA Lot A, PhtD Lot D Unadjuvanted, i.m. (100 μg/ml/protein)
L. PcpA Lot B, PhtD Lot D Unadjuvanted, i.m. (100 μg/ml/protein)
M. PcpA Lot A, PhtD Lot C+2 mM phosphate treated AlOOH, i.m. (100 μg/ml/protein)
N. PcpA Lot B, PhtD Lot C+2 mM phosphate treated AlOOH, i.m. (100 μg/ml/protein)
O. PcpA Lot A, PhtD Lot D+2 mM phosphate treated AlOOH, i.m. (100 μg/ml/protein)
P. PcpA Lot B, PhtD Lot D+2 mM phosphate treated AlOOH, i.m. (100 μg/ml/protein)

The bivalent formulations administered each included 5 μg/dose of each antigen (i.e., PhtD and PcpA) and were formulated with adjuvant in TBS pH 7.4 (1.3 mg/mL AlO(OH) pretreated with 2 mM phosphate). Mice were administered a lethal dose 1×10⁶ CFU) of S. pneumoniae strain MD, 4 days following the third (final) bleed.

Sample bleeds were taken from all animals one day prior to the first, second and third immunization and three weeks following the third immunization. Blood samples from individual mice were centrifuged at 9,000 rpm for 5 minutes and the recovered sera were stored at −20° C.

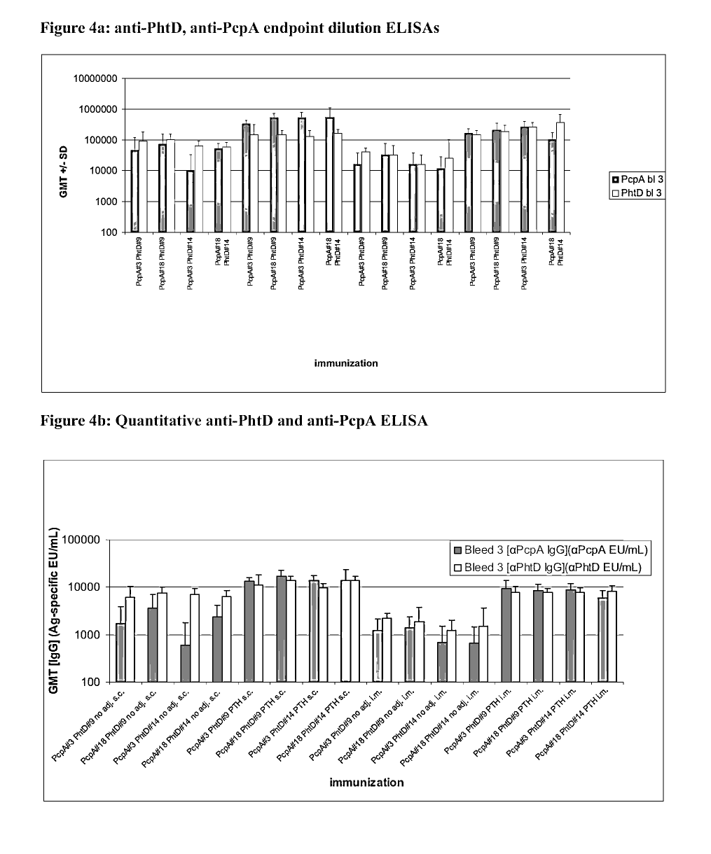

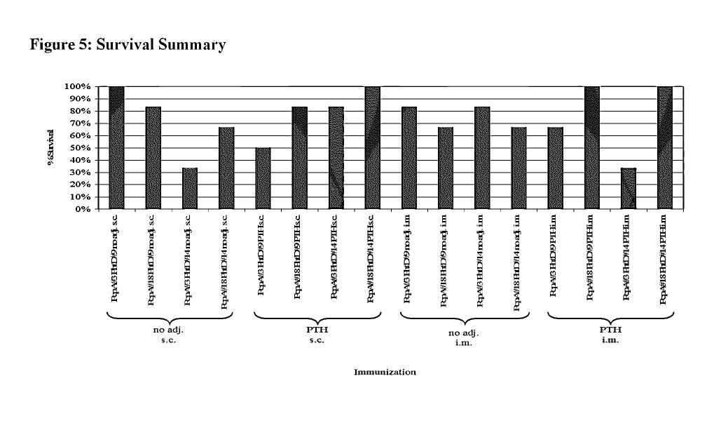

Total antigen-specific IgG titres were measured by endpoint dilution ELISA and by quantitative ELISA and geometric mean titres for each group are shown in FIGS. 4a to 4b. Survival results are summarized in FIG. 5.

There was no statistical difference between anti-PcpA and anti-PhtD IgG titres elicited by the different lots of PcpA and PhtD. There was an advantage noted in administering adjuvanted formulations subcutaneously; more specifically, formulations administered intramuscularly were less immunogenic than those administered subcutaneously. In addition, unadjuvanted formulations were less immunogenic than adjuvanted formulations.

In regards to survival, the formulations tested conferred protection against fatal S. pneumoniae challenge (100% survival seen in groups immunized with formulations of 100 μg/mL of each of PhtD and PcpA and pretreated AlO(OH)). There was no significant difference in % survival between the groups immunized intramuscularly and those immunized subcutaneously. The % survival of groups immunized with the two PhtD lots did not differ significantly whereas the % survival of groups immunized with the two PcpA lots did (with lot B providing a significantly higher survival). The PcpA lot B also gave significantly higher % survival in adjuvanted versus unadjuvanted formulations. There were no other statistical advantages noted in adjuvanted versus unadjuvanted formulations.

In this study, the particular lot of bacteria used for challenging the mice was found less virulent than a previously used lot of this bacterial strain. In a separate study (also using the intranasal challenge model), approximately 80% (p value 0.011) of the mice immunized with a formulation of 100 ug·mL of each of PhtD and PcpA+1.3 mg/mL AlO(OH) (Alhydrogel® “85” 2%, 25.08 mg/mL) in Tris-HCl, saline, 150 mM, at pH=7.4, survived a lethal S. pneumoniae challenge.

### Example 7

This Example describes the preparation of rabbit PhtD and PcpA anti-sera. Antisera were raised in rabbits using both His-tagged PhtD, His-tagged PcpA and recombinant PhtD and PcpA by a standard methodology. Measurement of PhtD and PcpA specific antibody in sera was determined by ELISA. As shown in Table 8, as an example for PhtD, a high titer of PhtD specific antibody was detected in the sera of all immunized rabbits but not in prebleed (before vaccination) sera. Both His-tagged PhtD and PhtD proteins were immunogenic in rabbits and antisera have high titres of PhtD specific antibody. Similar results were observed with His-PcpA and PcpA proteins (data not shown).

TABLE 8
Generation of PhtD Rabbit Antisera

| Study | Rabbit | Immunization | Bleed | ELISA Titers |
|---|---|---|---|---|
| 1 | 7 | His-tagged PhtD | pre-bleed | <100 |
| 1 | 7 | His-tagged PhtD | Final bleed | 409,600 |
| 1 | 8 | His-tagged PhtD | pre-bleed | <100 |
| 1 | 8 | His-tagged PhtD | Final bleed | 819,200 |
| 8 | 3 | PhtD | pre-bleed | <100 |
| 8 | 3 | PhtD | Final bleed | 819,200 |
| 8 | 4 | PhtD | pre-bleed | <100 |
| 8 | 4 | PhtD | Final bleed | 409,600 |

### Example 8

This Example describes the preparation of human PhtD and PcpA specific antibodies. Human polyclonal antibodies were purified from normal pooled adult human serum using affinity chromatography. Affinity chromatography columns were prepared using CNBr-activated sepharose resin covalently coupled to the purified recombinant antigen protein (PhtD or PcpA). Human AB serum (Sigma) was bound to the affinity column, which was then washed and the specific antibody eluted with Glycine-HCl buffer.

The final purified antibody was obtained by concentrating the pooled elution fractions by ultrafiltration and buffer exchange into PBS. The antibody solution was sterilized by filtration through a 0.22-μm syringe filter. The total protein concentration was determined using UV spectroscopy. The endotoxin level of the final antibody preparation was determined using an Endosafe PTS Reader from Charles River Laboratories. Purity, specificity and cross reactivity of the purified antibody was determined by SDS-PAGE, Western blot and antibody ELISA analysis. Each lot was purified from 100 mL of human AB serum unless otherwise stated.

### Example 9

### Surface Accessibility FACS Assay with Anti-PhtD and Anti-PcpA Antibodies

This Example describes the analysis of the binding capacity of anti-PhtD and anti-PcpA antibodies. Cultures were grown from frozen stocks to OD450 0.4-0.6, in either complete or Mn2+-depleted medium. Bacteria were washed and incubated with varying concentrations of human affinity purified antibodies in PBS. Human purified monoclonal antibodies against PspA were used as a positive control. Antibody binding to the bacteria was detected using a secondary antibody, FITC-conjugated anti-human IgG, and evaluated using flow cytometry. Similarly, anti-PhtD and anti-PcpA specific rabbit sera were used. Antibody binding to the bacteria was detected using a secondary antibody, FITC-conjugated anti-rabbit IgG and evaluated using flow cytometry.

As a qualitative assay read-out, bacteria were scored positive when a fluorescent signal was detected. Mean fluorescence intensity (MFI) was analyzed as a means of measuring the amount of antibodies bound to the surface of the bacteria.

Surface accessibility assays (SASSY') were performed to determine the ability of antigen-specific rabbit sera and purified human antibodies to bind live, intact S. pneumoniae.

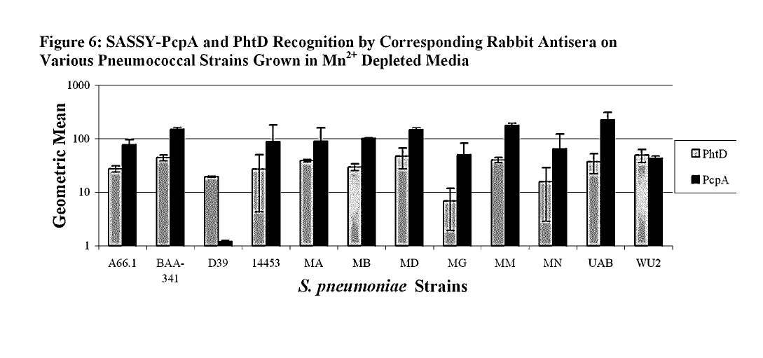

Purified human antibodies and rabbit PhtD- and PcpA-antisera (prepared as described in Example 7 and 8) bound protein on the surface of live S. pneumoniae. Both PhtD and PcpA rabbit antisera bound to all strains of S. pneumoniae tested, including laboratory and clinical isolates, with the exception of strain D39 which was negative for PcpA. However, this is consistent with the finding that strain D39 (a laboratory strain) was pcpA-negative by PCR amplification of the pcpA gene. In the case of PcpA, recognition occurred particularly when the bacteria were grown in conditions of depleted Mn2+ and increased Zn2+. Together, the data provide evidence that antibodies raised against recombinant protein or generated by natural infection recognize native protein and that epitopes on a wide variety of clinical isolates are conserved. The data also suggest that both PcpA and PhtD are highly surface accessible (FIG. 6, and data not shown). Rabbit preimmune sera were used as negative controls.

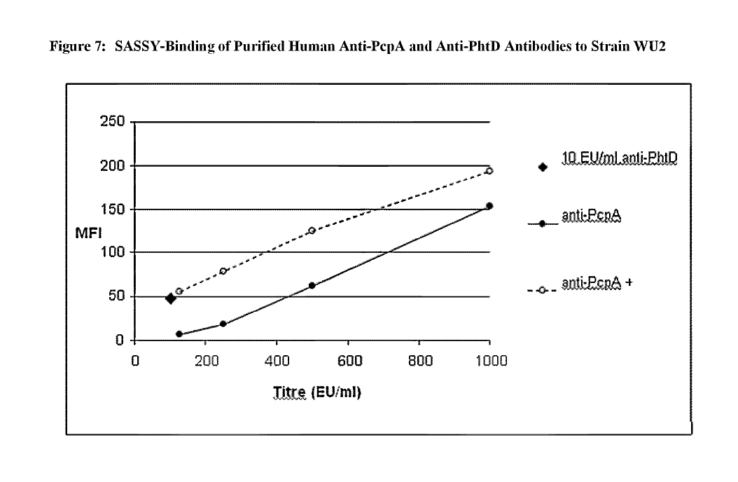

In order to determine whether human purified PhtD and PcpA antisera have any additive effects on binding to S. pneumoniae, 10 EU/ml anti-PhtD antibody was spiked into each sample containing increasing amounts of anti-PcpA antisera. The amount of total antibodies bound to the bacteria was measured by MFI (FIG. 7). Anti-PcpA antibodies were able to bind live S. pneumoniae in a dose-dependent manner. The addition of anti-PhtD antibodies led to a consistent increase in the MFI of the sample, confirming that antibodies against multiple surface proteins can bind simultaneously and that this leads to an increase in the total amount of antibody bound on the surface of the bacteria.

Purified human anti-PcpA antibodies, with or without purified human anti-PhtD antibodies, were incubated at varying concentrations with live S. pneumoniae strain WU2 which had been cultured in Mn2+-deficient medium. Antibodies bound to the surface of the bacteria were detected using FITC-goat-anti-human IgG. Mean Fluorescence Intensity (MFI) is shown in FIG. 7. Antibody titres are shown in anti-PcpA EU/ml (anti-PcpA and anti-PcpA+anti-PhtD samples) or anti-PhtD EU/ml (anti-PhtD sample).

Surface accessibility experiments with anti-PhtD and anti-PcpA rabbit sera and purified human antibodies indicated that both PcpA and PhtD are surface accessible. Furthermore, human anti-PcpA and anti-PhtD antibodies could bind simultaneously, and therefore, increase the total amount of antibodies bound to the bacteria.

### Example 10

This Example describes the analysis of the passive protection provided by a multivalent composition.

In this study, a bivalent composition of recombinant PhtD and PcpA formulated with AlPO₄ was used to immunize two New Zealand White Rabbits (Charles River) intramuscularly (i.m.) to obtain anti-PcpA/anti-PhtD polyclonal serum. Each rabbit was injected i.m. with 10 μg/dose of rPcpA and 10 μg/dose of rPhtD in AlPO₄ (3 mg/ml), (20 μg total protein, 500 μl total volume of injection/rabbit). Two subsequent immunizations were given at 3 week intervals with 10 μg/dose of rPcpA and 10 μg/dose of rPhtD in AlPO₄. Sample bleeds were collected following the 1^{st} and 2^{nd} immunizations. Final bleeds were collected three weeks following the final immunization. The blood was collected in gel separator tubes, allowed to clot, and serum was obtained by centrifugation, pooled and stored at about −20° C. The PhtD and PcpA-specific total IgG antibody titers were assessed for both rabbits. The serum from one of the rabbits used in the experiment had the following titer by ELISA: PhtD 204,800 and PcpA 102,400.

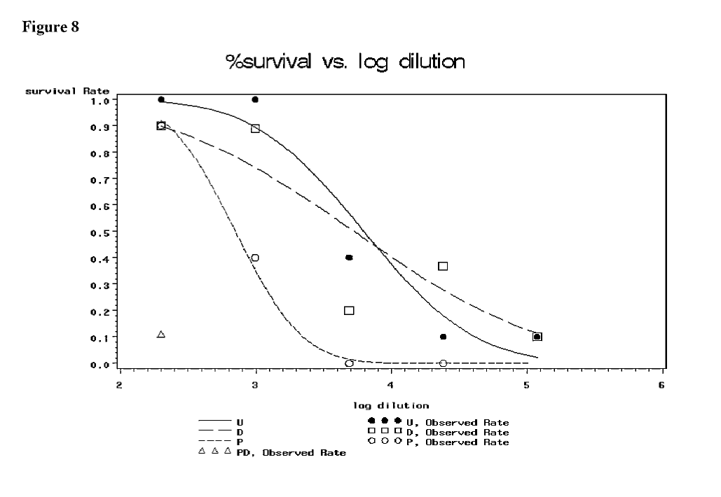

Recombinant PhtD protein and/or recombinant PcpA protein were added to certain sera samples to competitively inhibit (block) the corresponding antibodies present in the sera. As a control, neither recombinant protein was added to certain sera samples. Using a mouse model of passive protection based on one published earlier (Briles D E et. al., J. Infect Dis. 2000 December), various dilutions of sera samples were then administered to mice challenged with S. pneumoniae. The % survival observed per log dilution of sera administered was graphed in order to identify the Probit dose response curve (see FIG. 8). For each sera sample, the ED50 (log dilution effective for 50% survival) was calculated. Differences at ED50 between blocked and unblocked sera samples were assessed using a statistical model (see Table 9 below).

TABLE 9
Statistical Comparisons between protein blocked
groups to unblocked groups

| | Blocked | | 83% CI | 83% CI | |
|---|---|---|---|---|---|
| | Protein | ED50 | Low | High | Results |
| ED50 of 2-valent, | PcpA | 17 | 15 | 20 | S |
| unblocked | PhtD | 35 | 27 | 46 | NS |
| sera = 44 (36, 55) | Both PhtD and | — | — | — | S |
| | PcpA at 1:10* | | | | |
| Both PhtD and PcpA | PcpA | — | — | — | S |
| at 1:10* | | | | | |
| Both PhtD and PcpA | PhtD | — | — | — | S |
| at 1:10* | | | | | |

*Fisher's Exact Test

Competitively inhibiting the PcpA antibodies in the sera containing both PcpA and PhtD specific antibodies significantly decreased the ED50 (i.e., the log dilution of the sera effective for 50% survival) and this difference was statistically significant in comparison to the ED50 of unblocked sera. Competitively inhibiting the PhtD antibodies in the sera containing both PcpA and PhtD specific antibodies also decreased the ED50 (albeit not statistically significant). In regards to the sera sample in which both PcpA and PhtD antibodies were competitively inhibited (by adding to the sera each of PhtD and PcpA protein at a protein to sera ratio of 1:10), a low % survival was obtained with statistical significance by Fisher's Exact Test only with the highest dilution used and therefore ED50 was not determinable.

In sum, both the PhtD and PcpA antibodies contributed to the passive protection elicited by the sera raised to the bivalent formulation. The protection provided by the sera raised to the bivalent formulation was blocked by competitively inhibiting both PhtD and PcpA antibodies, and this result was significantly different from that obtained when only one of the antibodies (PhtD or PcpA) was competitively inhibited. Similar results were obtained using PhtD and PcpA proteins with rabbit trivalent hyper-immune sera (raised using a trivalent composition comprising PhtD, PcpA and PlyD1) in the same passive protection model. In that study, PhtD and PcpA proteins together were able to block the protective potential of the trivalent hyper-immune sera. These results from this passive protection model imply that the contributions of each protein-specific antibody are additive.

### Example 11

### Effects of Aluminum Concentration on Immunogenicity of Vaccine Formulation

This Example describes the analysis of the immunogenicity of a multi-component composition formulated with phosphate pretreated AlO(OH) and varying concentrations of elemental aluminum.

Female Balb/c mice were used to assess the immune response elicited by adjuvanted trivalent formulations. To prepare the trivalent formulations, recombinant PhtD, PcpA and an enzymatically inactive pneumolysin mutant (PlyD1, as described in PCT/CA/2009/001843, as SEQ ID NO:44 and herein as SEQ ID NO:9) were formulated with AlO(OH)-containing PO₄ (2 mM) as described in Example 1. Samples of prepared formulations were stored at 2 to 8° C. prior to the start of the study. Groups of Balb/c mice were immunized intramuscularly (IM) three times at 3 week intervals with the applicable formulation:

A. Unadjuvanted (Trivalent 50 μg/mL of PcpA and PhtD and 100 μg/mL of Ply mutant in TBS pH=7.4)

B. Trivalent 50 μg/mL of PcpA and PhtD and 100 μg/mL of Ply mutant+0.56 mg Al/mL PTH, P:Al molar ratio=0.1 (0.56 mg Al/mL AlO(OH) treated with 2 mM PO₄) in Tris Saline pH=7.4.

C. Trivalent 50 ug/mL of PcpA and PhtD and 100 ug/mL of Ply mutant+0.28 mg Al/mL PTH, P:Al molar ratio=0.1 (0.28 mg Al/mL AlO(OH) treated with 1 mM PO4) in Tris Saline pH=7.4.

D. Trivalent 50 ug/mL of PcpA and PhtD and 100 ug/mL of Ply mutant+1.12 mg Al/mL PTH, P:Al molar ratio=0.1 (1.12 mg Al/mL AlO(OH) treated with 4 mM PO₄) in Tris Saline pH=7.4.

E. Trivalent 50 μg/mL of PcpA and PhtD and 100 μg/mL of Ply mutant+1.68 mg Al/mL PTH, P:Al molar ratio=0.1 (1.68 mg Al/mL AlO(OH) treated with 6 mM PO4) in Tris Saline pH=7.4.

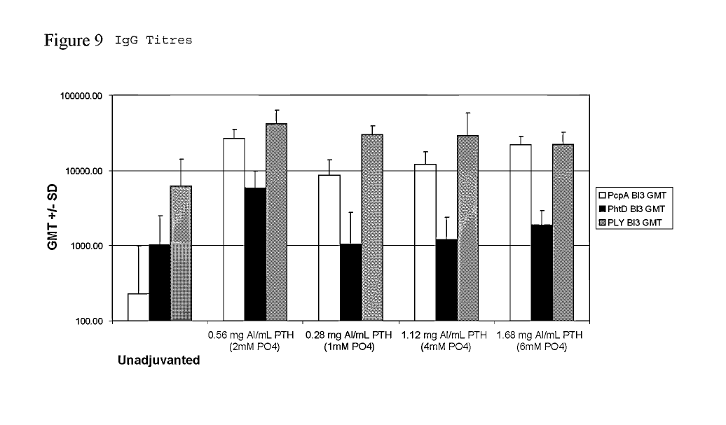

Sera were collected following the 1st, second and third immunization. Total antigen-specific IgG titres were measured by quantitative ELISA and geometric mean titres (+/−SD) for each group were calculated. A summary of the total IgG titers obtained are set out in FIG. 9.

All adjuvanted groups (B, C, D and E) produced significantly higher titres against all three antigens than the unadjuvanted group (A) (p<0.001). With respect to each antigen, titre levels peaked when adjuvanted with PTH with 0.56 mg elemental aluminum/mL (and, in the case of PhtD, the difference between titres elicited with aluminum 0.56 mg/mL and the two higher concentrations was statistically significant). Similarly, with respect to each antigen, titre levels were lower when adjuvanted with PTH with 0.28 mg elemental aluminum/mL (and, in the case of PcpA, the difference was statistically significant). These findings were surprising. Antibody (IgG) titers were expected to increase proportional to the concentration of aluminum (as reported in Little S. F. et. al., Vaccine, 25:2771-2777 (2007)). Surprisingly, even though the concentration of each of the antigens was kept constant, the titres decreased, rather than plateau, with increasing aluminum concentration (and with PhtD this was statistically significant).

### Example 12

This example describes the evaluation of the stability of an adjuvanted vaccine formulation under various conditions. A number of PTH adsorbed vaccine formulations were incubated for 5 days at 5° C., 25° C., 37° C. (i.e., under thermal accelerated conditions).

To evaluate the stability of 4 different vaccine formulations of PcpA (formulated in AlO(OH) or PTH), the formulations were each incubated for 6 weeks at 37° C. and then assessed by RP-HPLC. The stability results obtained are summarized in Table 10. The recovery from untreated AlO(OH) decreased by almost 50% following the incubation period (at 37° C.) whereas little to no degradation was observed in the PTH containing formulations.

TABLE 10
% Recovery (RP-HPLC) of PcpA after 6 weeks incubation at 37° C.

| | % Recovery | | % Adsorption | |
|---|---|---|---|---|
| | | T = 42 | | T = 42 |
| Formulation | T = 0 | days | T = 0 | days |
| 1) 50 μg/mL PcpA in 10 mM Tris-HCL, pH 7.4/150 mM | 98 | 53 | 100 | 100 |
| NaCl/1.3 mg/mL AlO(OH) | | | | |
| 2) 50 μg/mL PcpA in 10 mM Tris-HCl, pH 7.4/150 mM | 103 | 95 | 100 | 100 |
| NaCl/1.3 mg/mL AlO(OH)/2 mM Phosphate buffer pH 7.4 | | | | |
| 3) 50 μg/mL PcpA in 10 mM Tris-HCl, pH 7.4/150 mM | 103 | 98 | 100 | 100 |
| NaCl/1.3 mg/mL AlO(OH)/20 mM Phosphate buffer pH 7.4 | | | | |
| 4) 50 μg/mL PcpA in 10 mM Tris-HCl, pH 7.4/150 mM | 100 | 100 | 96 | 73 |
| NaCl/1.3 mg/mL AlO(OH)/80 mM Phosphate buffer pH 7.4 | | | | |

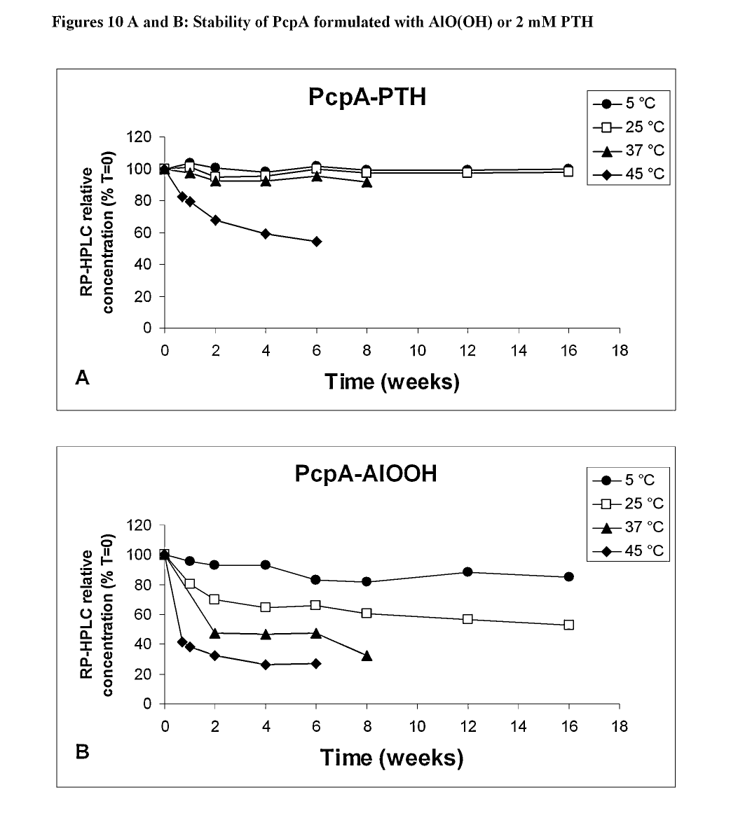

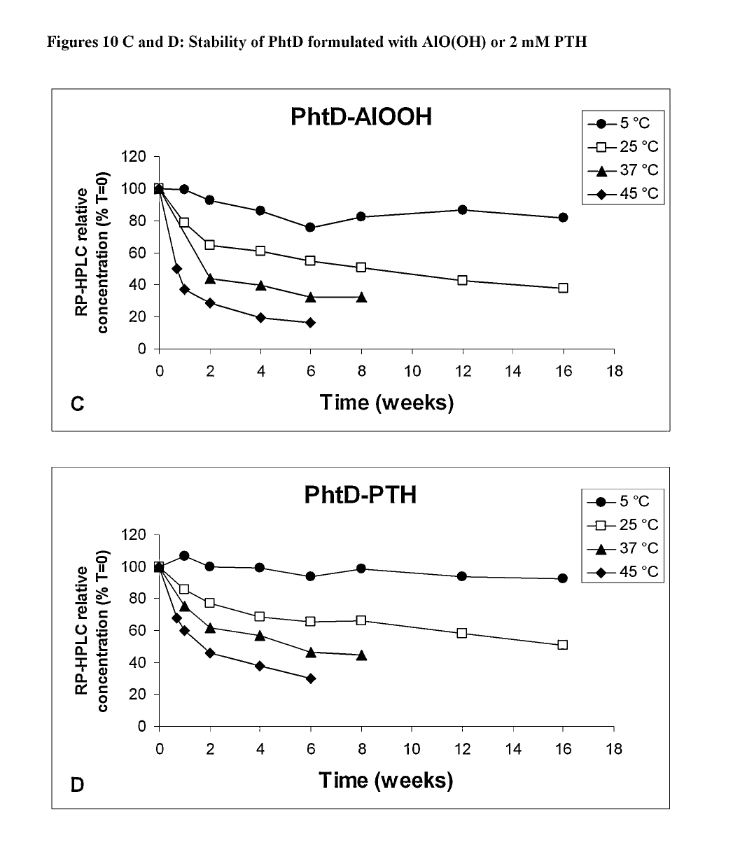

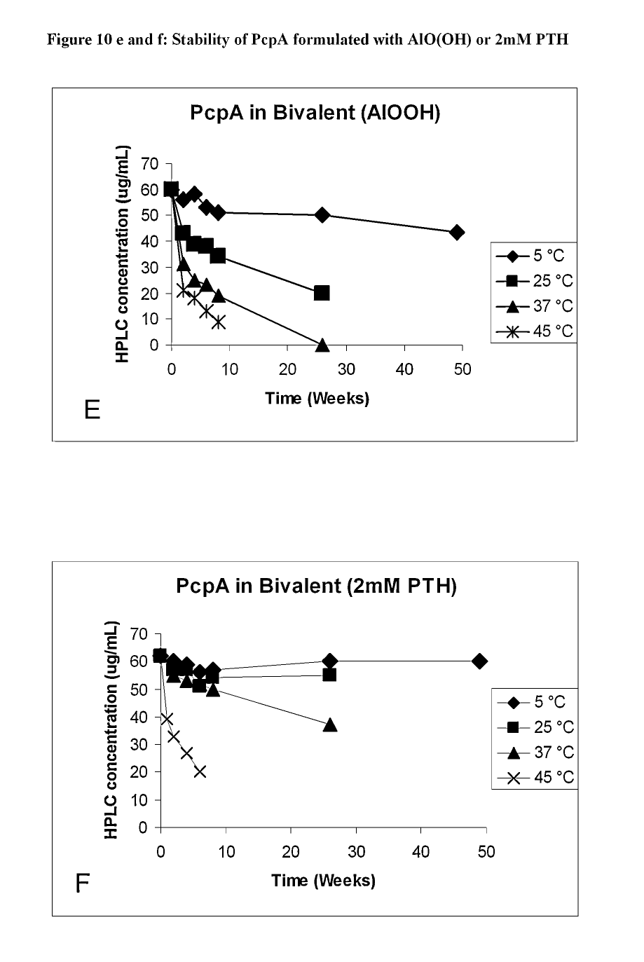

To evaluate the stability of PcpA and PhtD in monovalent and bivalent formulations (formulated with AlO(OH) or PTH), formulations were prepared as described in Example 1 using AlO(OH) or phosphate-treated AlO(OH) with 2 mM phosphate and samples were then incubated for about 16 weeks at various temperatures (i.e., 5° C., 25° C., 37° C. or 45° C.). Antigen concentration was then assessed by RP-HPLC. The stability results obtained are set out in FIGS. 10a to f. As shown the figures, in comparison to the formulations adjuvanted with untreated AlO(OH), the degradation rate of PcpA and PhtD, particularly under accelerated (stress) conditions (e.g., 25, 37, 45° C.) was significantly decreased in formulations adjuvanted with phosphate treated AlO(OH).

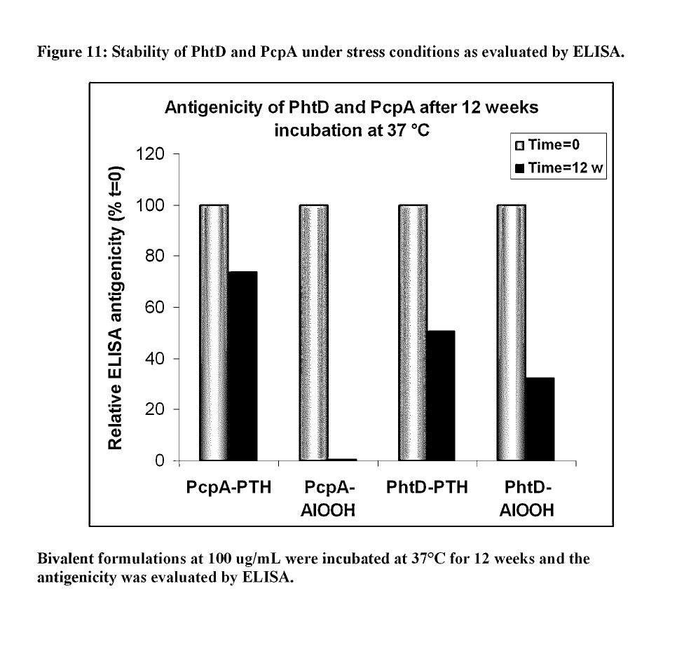

To evaluate to the antigenicity stability of the antigenicity of PcpA and PhtD in multi-valent formulations (formulated with AlO(OH) or PTH), bivalent formulations (at 100 μg/mL) were prepared as described in Example 1 and then samples were incubated at about 37° C. for approximately 12 weeks. Antigenicity of each formulation was evaluated by a quantitative ELISA sandwich assay at time zero and following the 12 week incubation period. Results are set out in FIG. 11. The antigenicity of both PcpA and PhtD following the 12 week incubation period at 37° C. was significantly higher when formulated with PTH in comparison to formulations with AlO(OH).

### Example 13

This example describes the evaluation of the effect of various excipients on the stability of a number of formulations.

A screening of 18 GRAS (generally regarded as safe) compounds at various concentrations was performed. An assay was used to screen for compounds that increase the thermal stability of each protein under evaluation (i.e., PcpA, PhtD and a detoxified pneumolysin mutant (PlyD1, as described in PCT/CA/2009/001843: Modified PLY Nucleic Acids and Polypeptides, as SEQ ID NO:44).

Each of the protein antigens were recombinantly expressed in E. coli and purified by serial column chromatography following conventional purification protocols substantially as described in Example 1, for PhtD and PcpA and as described in PCT/CA/2009/001843 (as SEQ ID NO:44) for PlyD1 (the sequence for which is noted herein as SEQ ID NO:9). Protein purity for all three antigens was typically higher than 90% as evaluated by RP-HPLC and SDS-PAGE. Proteins bulks were supplied at approximately 1 mg/mL in 10 mM Tris, pH 7.4 containing 150 mM sodium chloride. Each protein was diluted to the desired concentration (100 μg/mL PcpA; 100 μg/mL PhtD; 200 μg/mL PlyD1) with the appropriate excipient solution (in the concentration noted in Table 11) in 10 mM tris buffer saline, pH 7.5 (TBS), and PTH was added to the protein solutions to achieve a final concentration of 0.6 mg of elemental Al/mL. Control samples (lacking the applicable excipient) were also assayed. SYPRO® Orange, 5000× (Invitrogen, Inc., Carlsbad, Calif.), was diluted to 500× with DMSO (Sigma) and then added to the adjuvanted protein solutions. In all cases optimal dilution of SYPRO-Orange was 10× from a commercial stock solution of 5000×.

Assays were performed in a 96 well polypropylene plate (Stratagene, La Jolla, Calif.) using a real-time polymerase chain reaction (RT-PCR) instrument (Mx3005p QPCR Systems, Stratagene, La Jolla, Calif.). A sample volume of approximately 100 μL was added to each well and the plate was then capped with optical cap strips (Stratagene, La Jolla, Calif.) to prevent sample evaporation. Plates were centrifuged at 200 g for 1 min at room temperature in a Contifuge Stratos centrifuge (Heraeus Instruments, England) equipped with a 96 well plate rotor. The plates were then heated at 1° C. per min from 25° C. to 96° C. Fluorescence excitation and emission filters were set at 492 nm and 610 nm, respectively. Fluorescence readings (emission at 610 nm, excitation at 492 nm) were taken for each sample at 25° C. and then with each increase in 1° C.

Thermal transitions (melting temperatures, Tm) were obtained using the corresponding temperature of the first derivative of the minimum of fluorescence. The minimum of the negative first derivative trace from the melting curve (or dissociation curve) was calculated using MxPro software provided with RT-PCR system. Tm is defined as a midpoint in a thermal melt and represents a temperature at which the free energy of the native and non-native forms of a protein are equivalent. The effect of each excipient was assessed as the ΔTm=Tm (sample with protein+compound)−Tm (protein control sample). A summary of the results obtained are noted in Table 11. The sensitivity of the assay was +/−0.5° C.

Polyols, monosaccharides and disaccharides increased the Tm of adjuvanted PlyD1 in a concentration dependant manner with maximum stabilization (i.e., an increase in Tm of about 4° C.) observed at high concentration of sugars. Similar results were detected for each of PcpA and PhtD with the exception of arginine which decreased the Tm of PhtD by about 2° C. The following excipients were found to efficiently increase the thermal stability of all three proteins: sorbitol (20%, 10%), trehalose (20%), dextrose (20%, 10%), sucrose (10%, 5%), and 10% lactose.

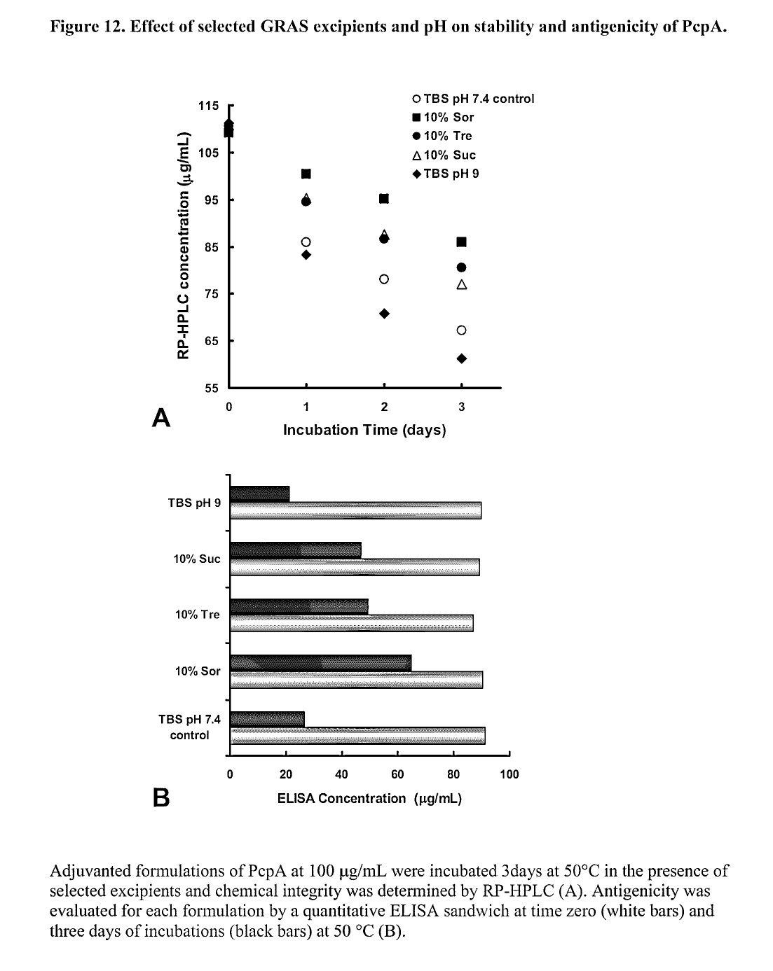

The effect of several excipients identified in the screening assays on the physical stability and antigenicity of PcpA stored under stress conditions was also studied to note any correlation with the thermal stability effects noted earlier. PcpA protein was diluted to the desired concentration (e.g., about 100 μg/mL) with the appropriate excipient solution described in the figure (10% Sorbitol, 10% Sucrose, 10% Trehalose in 10 mM Tris Buffer pH 7.4), and PTH was added to the protein solutions to achieve a final concentration of 0.6 mg of elemental Al/mL. A control sample (lacking excipient) was also included in the study. Samples were stored at 50° C. for a three day period. Protein degradation was evaluated by RP-HPLC and antigenicity was assessed by quantitative, sandwich ELISA. Results are set out in FIGS. 12A and 12B.

The concentration of intact protein was measured by RP-HPLC in an Agilent 1200 HPLC system equipped with a diode array UV detector. Samples were desorbed from the adjuvant in PBS/Zwittergent buffer for 5 h at 37° C. and separated using an ACE C4 column (Advanced Chromatography Technologies, Aberdeen, UK) and a mobile phase gradient of buffer A (0.1% TFA in water) and buffer B (0.1% TFA in CAN) using a gradient of 0.75% of buffer B per minute over 30 min at a flow rate of 1 ml/min. Proteins were monitored by UV absorbance at 210 nm and quantitated against a 5-point linear calibration curve produced with external standards.

The quantitative antigen ELISA sandwich was used to evaluate antigenicity of PcpA formulations at time zero and after 3 days of incubation at 50° C. A rabbit IgG anti-PcpA sera was used for antigen capture, and a well characterized monoclonal anti-PcpA for detection. Briefly, 96 well plates were coated with rabbit anti-PhtD IgG at a concentration of 2 μg/mL in 0.05M Na₂CO₃/NaHCO₃ buffer for 18 hours at room temperature (RT), and blocked with 1% BSA/PBS for 1 hour at RT followed by 2 washes in a washing buffer of PBS/0.1% Tween 20 (WB). Two-fold dilutions of test samples, an internal control and a reference standard of purified PcpA of known concentration were prepared in 0.1% BSA/PBS/0.1% Tween 20 (SB), added to wells and incubated at RT for 1 hour followed by 5 washes in WB. Detecting primary mAb was diluted in SB to a concentration of 0.1 μg/mL, and incubated for 1 hour at RT and followed by 5 washes in WB, and addition of F(ab′)2 Donkey anti-mouse IgG (H+L) specific at 1/40K dilution in SB. Following 5 washes in WB, TMB/H₂O₂ substrate is added to the wells, and incubated for 10 minutes at RT. The reaction is stopped by the addition of 1M H₂SO₄. ELISA plates were read in a plate reader (SpectraMax, M5, Molecular Devices, Sunnyvale, Calif.) at A450/540 nm, and test sample data is calculated by extrapolation from a standard curve using 4-parameter logistic using the software SoftMax PRO.

As shown in FIG. 12A, data derived from RP-HPLC showed that those excipients that increased the Tm of adjuvanted PcpA also decreased the protein's rate of degradation at 50° C. over a three day period. The greatest stability as determined by percent recovery of the PcpA protein over time was provided by 10% sorbitol (as shown in FIG. 12A). The antigenicity of adjuvanted PcpA was also preserved by these excipients (as shown in FIG. 12B). In good correlation with RP-HPLC results, sorbitol appeared to preserve antigenicity to a higher degree than sucrose or trehalose.

The addition of 10% sorbitol, 10% sucrose, or 10% trehalose significantly decreased the rate constant at 50° C. and increased the half life of PcpA when compared to that of the control sample without excipients (Table 12). The buffer pH of 9.0 decreased the Tm of the protein, but accelerated degradation (i.e., increased the rate constant) at 50° C. as compared to that of the control (Table 12). Altogether, these results suggest a good correlation between thermal stability detected by the assay, physical stability detected by RP-HPLC and antigenicity detected by ELISA.

In view of the results obtained in these studies, sorbitol, sucrose, dextrose, lactose and/or trehalose are examples of excipients that may be included in monovalent and multivalent (e.g., bivalent, trivalent) formulations of PcpA, PhtD and detoxified pneumolysin proteins (such as, PlyD1) to increase physical stability.

TABLE 11
Effect of GRAS excipients on Tm (as assessed by monitoring fluorescence emission over a
temperature range). Compounds that increase thermal stability provide a positive Tm difference value.

| | PcpA | | PhtD | | Ply mutant | |
|---|---|---|---|---|---|---|
| | | ΔTm | | ΔTm | | ΔTm |
| | | (ΔTm = Tm | | (ΔTm = Tm | | (ΔTm = Tm |
| | | (excipient) − | | (excipient) − | | (excipient) − |
| Excipient | Tm (° C.) | Tm (control) | Tm (° C.) | Tm (control) | Tm (° C.) | Tm (control) |
| Control | 56.7 | 0.0 | 58.7 | 0.0 | 49.7 | 0.0 |
| 5% Sucrose | 57.0 | 0.3 | 60.0 | 1.3 | 50.4 | 0.7 |
| 10% Sucrose | 58.4 | 1.7 | 60.0 | 1.3 | 52.1 | 2.4 |
| 20% Sucrose | 60.0 | 3.3 | 61.7 | 3.0 | 52.5 | 2.8 |
| 5% Dextrose | 57.7 | 1.0 | 58.7 | 0.0 | 49.7 | 0.0 |
| 10% Dextrose | 58.7 | 2.0 | 59.7 | 1.0 | 51.7 | 2.0 |
| 20% Dextrose | 60.7 | 4.0 | 60.7 | 2.0 | 53.7 | 4.0 |
| 5% Trehalose | 56.7 | 0.0 | 58.7 | 0.0 | 49.7 | 0.0 |
| 10% Trehalose | 57.7 | 1.0 | 58.7 | 0.0 | 50.7 | 1.0 |
| 20% Trehalose | 58.7 | 2.0 | 60.7 | 2.0 | 51.7 | 2.0 |
| 5% Mannitol | 56.7 | 0.0 | 58.7 | 0.0 | 49.7 | 0.0 |
| 10% Mannitol | 56.7 | 0.0 | 58.7 | 0.0 | 49.7 | 0.0 |
| 20% Mannitol | 56.7 | 0.0 | 58.7 | 0.0 | 50.7 | 1.0 |
| 5% Sorbitol | 56.7 | 0.0 | 58.7 | 0.0 | 49.7 | 0.0 |
| 10% Sorbitol | 58.7 | 2.0 | 59.7 | 1.0 | 51.7 | 2.0 |
| 20% Sorbitol | 60.7 | 4.0 | 60.7 | 2.0 | 53.7 | 4.0 |
| 5% Glycerol | 56.7 | 0.0 | 58.7 | 0.0 | 49.7 | 0.0 |
| 10% Glycerol | 56.7 | 0.0 | 58.7 | 0.0 | 49.7 | 0.0 |
| 20% Glycerol | 56.7 | 0.0 | 58.7 | 0.0 | 49.7 | 0.0 |
| 0.05M Lysine | 56.7 | 0.0 | 58.7 | 0.0 | 49.7 | 0.0 |
| 0.1M Lysine | 56.7 | 0.0 | 58.7 | 0.0 | 49.7 | 0.0 |
| 5% Lactose | 56.7 | 0.0 | 58.7 | 0.0 | 50.7 | 1.0 |
| 10% Lactose | 58.7 | 2.0 | 60.7 | 2.0 | 50.7 | 1.0 |
| 0.05M Proline | 56.7 | 0.0 | 58.7 | 0.0 | 48.7 | −1.0 |
| 0.1M Proline | 56.7 | 0.0 | 58.7 | 0.0 | 48.7 | −1.0 |
| 0.05M Glycine | 56.7 | 0.0 | 58.7 | 0.0 | 50.7 | 1.0 |
| 0.1M Glycine | 56.7 | 0.0 | 58.7 | 0.0 | 50.7 | 1.0 |
| 0.01M Aspartate | 56.7 | 0.0 | 58.7 | 0.0 | 48.7 | −1.0 |
| 0.05M Glutamate | 56.7 | 0.0 | 58.7 | 0.0 | 50.7 | 1.0 |
| 0.05M Lactic acid | 56.7 | 0.0 | 58.7 | 0.0 | 49.7 | 0.0 |
| 0.05M Malic Acid | 58.7 | 2.0 | 58.7 | 0.0 | 48.7 | −1.0 |
| 0.05M Arginine | 56.7 | 0.0 | 58.7 | 0.0 | 48.7 | −1.0 |
| 0.1M Arginine | 56.7 | 0.0 | 56.7 | −2.0 | 48.7 | −1.0 |
| 0.05M Diethanolamine | 56.7 | 0.0 | 58.7 | 0.0 | 48.7 | −1.0 |
| 0.1M Diethanolamine | 56.7 | 0.0 | 58.7 | 0.0 | 48.7 | −1.0 |
| 0.05M Histidine | 56.7 | 0.0 | 58.7 | 0.0 | 50.7 | 1.0 |
| 0.1M Histidine | 56.7 | 0.0 | 58.7 | 0.0 | 49.7 | 0.0 |
| 0.15M Taurine | 56.7 | 0.0 | 58.7 | 0.0 | 50.7 | 1.0 |

TABLE 12
Rate constant values from stability data of
formulations incubated at 50° C.

| | k at 50° C. | Half life at 50° C. | |
|---|---|---|---|
| Formulation | (μg · mL⁻¹ · day⁻¹) | (days) | R² |
| 10% Sorbitol | 7.5 | 7.3 | 0.99 |
| 10% Trehalose | 9.8 | 5.6 | 0.95 |
| 10% Sucrose | 10.9 | 5.1 | 0.98 |
| Control (TBS pH 7.4) | 13.4 | 4.1 | 0.94 |
| TBS pH9 | 16.2 | 3.4 | 0.93 |

Rate constant for formulations incubated at 50° C. were calculated by fitting the RP-HPLC stability data presented in Figure 12A using zero order kinetics equation (1) [A₁] = − kt + [A₀], where Aₜ is the concentration of the antigen at a given time, A₀ is the initial protein concentration in μg/mL and t is the time in days. R² is reported for the linear fit of the data using equation (1).

### Example 14

The effect of pH on the stability of three different antigens formulated with or without an aluminum adjuvant was performed. An assay was used to evaluate the effect of pH on the thermal stability of each protein under evaluation (i.e., PcpA, PhtD and a detoxified pneumolysin mutant (PlyD1, as described in PCT/CA2009/001843: Modified PLY Nucleic Acids and Polypeptides, as SEQ ID NO:44 and noted in the Sequence Listing herein as SEQ ID NO:9).

Each of the protein antigens were recombinantly expressed in E. coli and purified by serial column chromatography following conventional purification protocols substantially as described in Example 1, for PhtD and PcpA and as described in PCT/CA2009/001843 for PlyD1. Protein purity for all three antigens was typically higher than 90% as evaluated by RP-HPLC and SDS-PAGE. Proteins bulks were supplied at approximately 1 mg/mL in 10 mM Tris, pH 7.4 containing 150 mM sodium chloride. Each protein was diluted to the desired concentration (100 μg/mL PcpA; 100 μg/mL PhtD; 200 μg/mL PlyD1) with the appropriate buffer solution (i.e., 10 mM Tris buffer (pH 7.5-9.0), 10 mM phosphate buffer (pH 6.0-7.0) and 10 mM acetate buffer (pH 5.0-5.5)) and an aluminum adjuvant (i.e., aluminum hydroxide (Alhydrogel, Brenntag Biosector, Denmark), or aluminum phosphate (Adju-Phos, Brenntag Biosector, Denmark) or aluminum hydroxide pre-treated with 2 mM phosphate (PTH)) was added to the protein solutions to achieve a final concentration of 0.6 mg of elemental Al/mL. Control samples (lacking the applicable adjuvant) were also assayed. SYPRO® Orange, 5000× (Invitrogen, Inc., Carlsbad, Calif.), was diluted to 500× with DMSO (Sigma) and then added to the adjuvanted protein solutions. In all cases optimal dilution of SYPRO-Orange was 10× from a commercial stock solution of 5000×.

Assays were performed in a 96 well polypropylene plate (Stratagene, La Jolla, Calif.) using a real-time polymerase chain reaction (RT-PCR) instrument (Mx3005p QPCR Systems, Stratagene, La Jolla, Calif.). A sample volume of approximately 100 μL was added to each well and the plate was then capped with optical cap strips (Stratagene, La Jolla, Calif.) to prevent sample evaporation. Plates were centrifuged at 200 g for 1 min at room temperature in a Contifuge Stratos centrifuge (Heraeus Instruments, England) equipped with a 96 well plate rotor. The plates were then heated at 1° C. per min from 25° C. to 96° C. Fluorescence excitation and emission filters were set at 492 nm and 610 nm, respectively. Fluorescence readings (emission at 610 nm, excitation at 492 nm) were taken for each sample at 25° C. and then with each increase in 1° C.

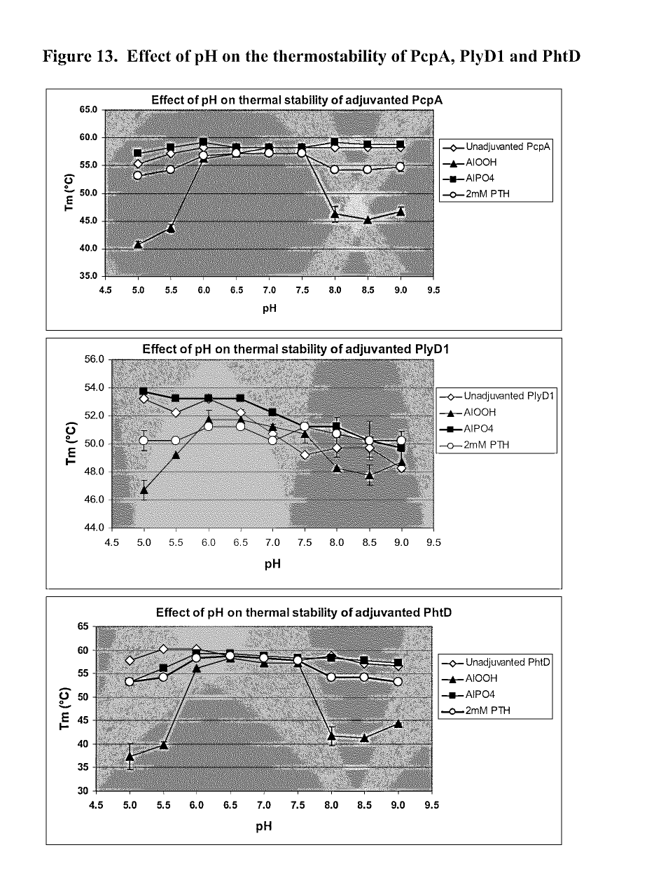

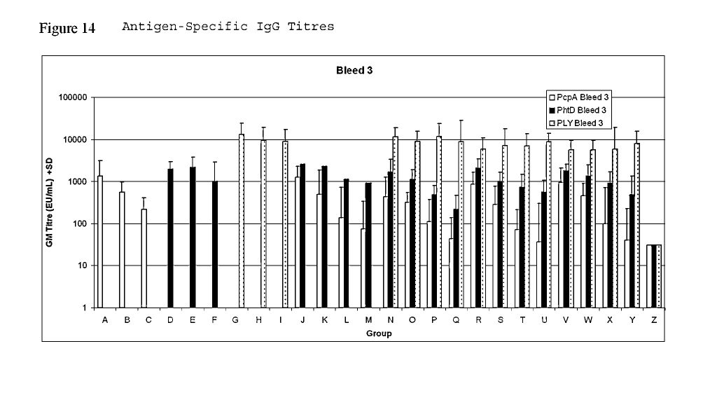

Thermal transitions (melting temperatures, Tm) were obtained using the corresponding temperature of the first derivative of the minimum of fluorescence. The minimum of the negative first derivative trace from the melting curve (or dissociation curve) was calculated using MxPro software provided with RT-PCR system. Tm is defined as a midpoint in a thermal melt and represents a temperature at which the free energy of the native and non-native forms of a protein are equivalent. A summary of the results obtained are noted in FIG. 13. The sensitivity of the assay was +/−0.5° C.

For most proteins, solution pH determines the type and total charge on the protein, and thus, may affect electrostatic interactions and overall stability. For adjuvanted proteins the solution pH and buffer species have a strong effect on microenvironment pH at the surface of the aluminum adjuvants which could ultimately influence the degradation rate of proteins adsorbed to aluminum adjuvants.

All three proteins were 90 to 100% adsorbed to aluminum hydroxide in the range of pH under study. In aluminum phosphate, the adsorption of PcpA was higher than 80% while PhtD and PlyD1 (each an acidic protein) were negligibly adsorbed to the adjuvant above pH 5 (data not shown).

FIG. 13 shows the effect of pH on each of the 3 antigens when formulated with adjuvant and in unadjuvanted controls. The unadjuvanted antigens displayed their distinctive pH stability profile. PcpA showed steady Tm values on a broad pH range from 6.0 to 9.0 with decreasing Tm values as the pH was dropped from 6.0 to 5.0. On the other hand, the thermal stability of unadjuvanted PhtD and PlyD1 appeared maximized under acidic pHs (see FIG. 13). The thermal stability profiles of the unadjuvanted proteins were significantly modified as a result of the addition of an aluminum adjuvant. As compared to the unadjuvanted controls, aluminum hydroxide, appeared to decrease the stability of all three proteins at relatively high and low pH values showing a bell-shaped curve as the pH was increased from 5 to 9 with a maximum stability at near neutral pH. These data show that pretreatment of AlOOH with 2 mM phosphate significantly improved the stability of all three antigens at high and low pH as compared to untreated AlOOH (FIG. 13 A-C). No significant changes were observed in the range of pH 6.0-7.5 by this method.

As compared to unadjuvanted controls, no major changes were observed on the Tm vs pH profile of PcpA and PlyD1 when aluminum phosphate was used as the adjuvant (FIGS. 13A and 13C). In the case of PhtD adjuvanted with AP, as compared to the unadjuvanted control, a significant decrease in the Tm was observed at pH lower than 6 (FIG. 13B).

### Example 15

This example describes the evaluation of the effect of various antigen combinations in multi-component formulations.

Three separate S. pneumoniae antigens were formulated in monovalent, bivalent and trivalent form and evaluated using the IN challenge model (substantially as described in previous examples). Monovalent, bivalent and trivalent formulations were prepared using suboptimal doses of purified recombinant PcpA, PhtD and PlyD1 (a detoxified pneumolysin) in TBS with adjuvant (AlOOH treated with 2 mM PO₄ (0.56 μg Al/dose)) pH 7.4. Suboptimal doses of each antigen that had been shown to induce either limited or no protection were chosen so as to detect additive effects. Each of the protein antigens were recombinantly expressed in E. coli and purified by serial column chromatography following conventional purification protocols substantially as described earlier. Protein purity for all three antigens was typically higher than 90% as evaluated by RP-HPLC and SDS-PAGE. Groups (n=26) of female CBA/J mice (n=15/group) were immunized intramuscularly three times at 3 week intervals between each immunization with applicable formulations (504).

Mice were administered a lethal dose of S. pneumoniae strain 14453, serotype 6B (1.5×10⁶ cfu/mouse 3 weeks post final immunization and observed for survival and health for 2 weeks. Survival results (summarized in Table 13 below) were calculated and statistically analyzed by Fisher Exact test. Total antigen-specific IgG titres (from sera that had been collected following each immunization) were measured by quantitative ELISA and geometric mean titres (+/−SD) for each group were calculated. A summary of the total IgG titers obtained are set out in FIG. 14.

TABLE 13

| Group/ | | | | | Significant |
|---|---|---|---|---|---|
| Formulation | PcpA | PhtD | PlyD1 | | protection Fisher |
| administered | (μg/50 μl) | (μg/50 μl) | (μg/50 μl) | % Survival | Exact test |
| A/Monovalent | 0.06 | | | 73.333333 | + |
| B/Monovalent | 0.02 | | | 66.666667 | + |
| C/Monovalent | 0.0067 | | | 66.666667 | + |
| D/Monovalent | | 0.25 | | 20 | − |
| E/Monovalent | | 0.083 | | 26.666667 | − |
| F/Monovalent | | 0.027 | | 33.333333 | − |
| G/Monovalent | | | 0.5 | 46.666667 | − |
| H/Monovalent | | | 0.166 | 13.333333 | − |
| I/Monovalent | | | 0.055 | 33.333333 | − |
| J/Bivalent | 0.06 | 0.25 | | 73.333333 | + |
| K/Bivalent | 0.02 | 0.083 | | 66.666667 | + |
| L/Bivalent | 0.0067 | 0.027 | | 33.333333 | − |
| M/Bivalent | 0.00335 | 0.0135 | | 40 | − |
| N/Trivalent | 0.06 | 0.25 | 0.5 | 90.909091 | + |
| O/Trivalent | 0.02 | 0.083 | 0.5 | 73.333333 | + |
| P/Trivalent | 0.0067 | 0.027 | 0.5 | 73.333333 | + |
| Q/Trivalent | 0.00335 | 0.0135 | 0.5 | 40 | − |
| R/Trivalent | 0.06 | 0.25 | 0.166 | 70 | + |
| S/Trivalent | 0.02 | 0.083 | 0.166 | 80 | + |
| T/Trivalent | 0.0067 | 0.027 | 0.166 | 73.333333 | + |
| U/Trivalent | 0.00335 | 0.0135 | 0.166 | 26.666667 | − |
| V/Trivalent | 0.06 | 0.25 | 0.055 | 69.230769 | + |
| W/Trivalent | 0.02 | 0.083 | 0.055 | 86.666667 | + |
| X/Trivalent | 0.0067 | 0.027 | 0.055 | 60 | + |
| Y/Trivalent | 0.00335 | 0.0135 | 0.055 | 46.666667 | − |
| Z/Placebo Control | | | | 20 | − |

The PcpA monovalent formulations were protective even at very low doses (and despite low antibody titres). In comparison to the PcpA monovalent formulation, the trivalent formulations provided similar levels of protection. In comparison to the PhtD and PlyD1 monovalent formulations, the trivalent formulations provided significantly higher protection. The trivalent formulations elicited higher survival percentages as compared to the bivalent formulations (and difference was statistically significant, p=0.043, in regards to two trivalent formulations (0.0067:0.027:0.5; 0.0067:0.027:0.166; PcpA:PhtD:PlyD1) in comparison to bivalent formulation (0.0067:0.027; PcpA:PhtD)). The bivalent formulation was not protective at 0.0067 and 0.027 μg for PcpA and PhtD, respectively, which for PcpA was a protective dose when administered as a monovalent formulation. However, as the difference in survival between these two groups was not statistically significant, the observed difference between monovalent/bivalent formulations was due to assay variability.

The median effective dose of each of PcpA and PhtD in protecting at least 60% of mice from lethal challenge (ED60) in a bivalent formulation (0.0067:0.027; PcpA:PhtD) and in the trivalent formulations were calculated (see Table 14 below). For each of PcpA and PhtD, the ED60 was reduced in the trivalent formulations as compared to the corresponding bivalent formulation. By these results, the addition of PlyD1 had on average a 2-fold dose sparing effect on the bivalent formulation (i.e., PcpA+PhtD).

These data show that immunization with trivalent formulations elicits better protection as compared to bivalent formulations. The inclusion of PlyD1 in the trivalent formulations does not have an inhibitory effect on overall protection.

TABLE 14

| | | | | | | | Fold |
|---|---|---|---|---|---|---|---|
| | | | | | | | decrease in |
| Group | PcpA | | | PhtD | | | dose |
| PcpA:PhtD:PlyD1 | | 83% CI | | | 83% CI | | compared to |
| (μg in 50 μL) | ED60 | low | high | ED60 | Low | High | bivalent |
| L (PcpA:PhtD = | 0.014 | 0.0085 | 0.0234 | 0.0567 | 0.0341 | 0.0943 | |
| 0.0067:0.027) | | | | | | | |
| P (PcpA:PhtD:PlyD1 = | 0.0067 | 0.0041 | 0.0108 | 0.0269 | 0.0167 | 0.0434 | 2.105 |
| 0.0067:0.027:0.5) | | | | | | | |
| T (PcpA:PhtD:PlyD1 = | 0.0074 | 0.0046 | 0.0119 | 0.0297 | 0.0185 | 0.0478 | 1.907 |
| 0.0067:0.027:0.166) | | | | | | | |
| X (PcpA:PhtD:PlyD1 = | 0.0058 | 0.0036 | 0.0095 | 0.0236 | 0.0145 | 0.0383 | 2.404 |
| 0.0067:0.027:0.055) | | | | | | | |

### Example 16

This example describes the evaluation of the minimum effective antigen dose that elicits the highest level of antibody responses.

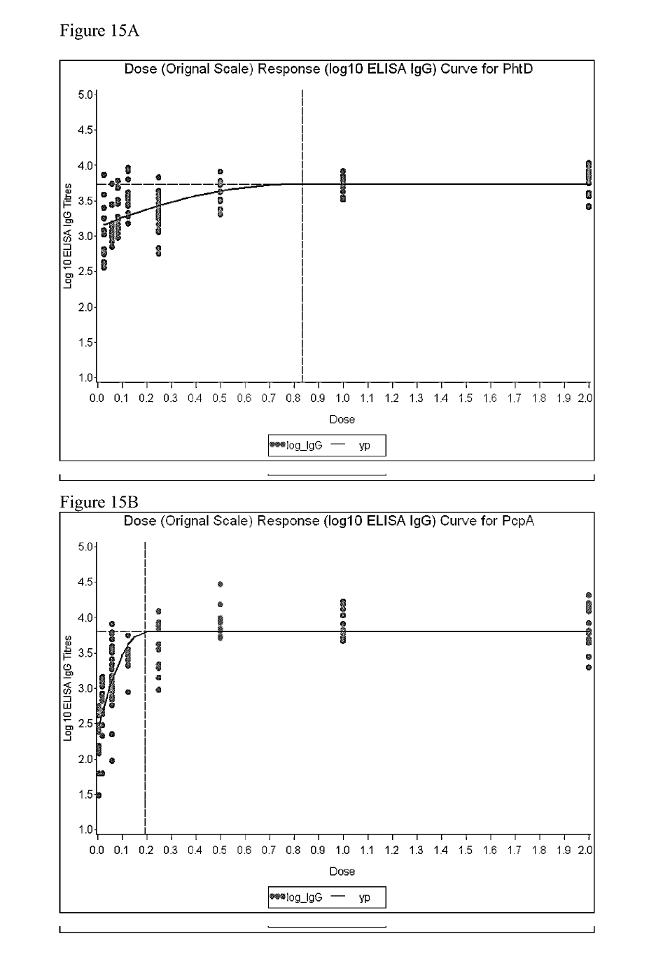

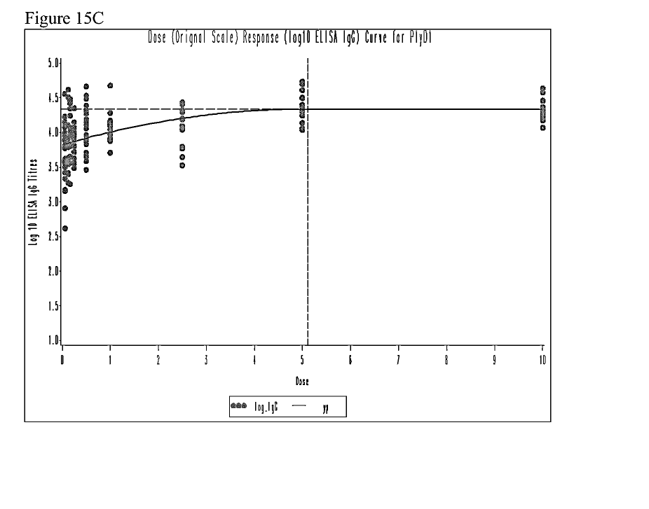

From monovalent studies conducted total antigen-specific IgG titres (as measured by ELISA) per antigen dose were graphically plotted to evaluate the minimum effective antigen dose eliciting highest titre. Representative graphs are set out in FIGS. 15 A, B, C. For PcpA, the estimated minimum antigen dose was assessed as 0.196 μg/mouse (0.147, 95% low; 0.245, 95% high), and for PhtD the estimated minimum antigen dose was assessed as 0.935 μg/mouse (0.533, 95% low; 1.337, 95% high) which provides a ratio of PcpA:PhtD of 1:4. The minimum antigen dose for PlyD1 was estimated as >5 μg/mouse. As no immunological interference between antigens were detected at any of the evaluated ratios in the bivalent and trivalent studies performed (such as, for example, in Example 15), a 1:1:1 ratio of PcpA:PhtD:PlyD1 may be used in a muli-component composition.

### REFERENCES

1. Henrichsen J. Six newly recognized types of Streptococcus pneumoniae.
2. Park I H, Pritchard D G, Cartee R et al. 2007. Discovery of a new capsular serotype (6C) within serogroup 6 of Streptococcus pneumoniae. J. Clin. Microbiol. 45, 1225-1233.
3. World Health Organization. 2007. Pneumococcal conjugate vaccine for childhood immunization—WHO position paper. Wkly Epidemiol. Rec. 82, 93-104.
4. Plotkin, S. A. and Orenstein W. A. Vaccines. Editors W. B. Saunders Company, Third Edition 1999
5. Fedson, D. S. et al, (1999), The burden of pneumococcal disease among adults in developed and developing countries: what is known and what is not known. Vaccine 17, S11-S18.
6. Klein, D. L. (1999) Pneumococcal disease and the role of conjugate vaccines. Microb. Drug Resist., 5, 147-157.
7. Rahav, G., et al, (1997) Invasive pneumococcal infection: A comparison between adults and children. Medicine 76, 295:303.
8. World Health Organization Bulletin 2004. Global estimate of the incidence of clinical pneumonia among children under five years of age. December 2004, 82 (12).
9. Siber G R, Klugman K P, Makela P H. Pneumococcal Vaccines: The Impact of Conjugate Vaccine. Washington D.C.: ASM Press; 2008
10. PREVNAR® (package insert). Wyeth Pharmaceuticals Inc. Philadelphia, Pa. 2006
11. Clinical and Vaccine Immunology, June 2007, p. 792-795; Pediatr. Infect. Dis. J. 16(4 Suppl.):S97-S102.
12. WHO (2005). Guidelines on nonclinical evaluations vaccines. Technical report series No. 927.

1. An immunogenic composition comprising an isolated immunogenic S. pneumoniae PcpA polypeptide and an isolated immunogenic S. pneumoniae polypeptide selected from the group consisting of the polyhistidine triad family of proteins.

2. An immunogenic composition of claim 1 for conferring protection in a subject against disease caused by S. pneumoniae infection which comprises an isolated immunogenic S. pneumoniae PcpA polypeptide and an isolated immunogenic S. pneumoniae polypeptide selected from the group consisting of the polyhistidine triad family of proteins.

3. The composition of claim 1 wherein the composition comprises an isolated immunogenic S. pneumoniae PcpA polypeptide and an isolated immunogenic S. pneumoniae PhtD polypeptide or a fusion protein thereof.

4. The composition of claim 3 wherein the amino acid sequence of the PhtD polypeptide has at least 80% sequence identity to the amino acid sequence as set forth in SEQ ID NO:1.

5. The composition of claim 3 wherein the PhtD polypeptide is produced recombinantly.

6. The composition of claim 5 wherein the recombinantly produced PhtD polypeptide is an N-terminal truncation lacking the signal peptide sequence.

7. The composition of claim 3 wherein the PhtD protein comprises a polypeptide having an amino acid sequence that has at least 80% sequence identity to the amino acid sequence as set forth in SEQ ID NO:5 and/or the PcpA polypeptide has at least 80% sequence identity to the amino acid sequence as set forth in SEQ ID NO:2 or SEQ ID NO:7.

8-14. (canceled)

15. The composition of claim 3 comprising:
about 5 to 100 μg/dose of the PhtD polypeptide and
about 5 to 100 μg/dose of the PcpA polypeptide.

16. The composition of claim 1 wherein the composition further comprises pneumolysin.

17. The composition of claim 16 wherein the pneumolysin is detoxified.

18. The composition of claim 17 wherein the detoxified pneumolysin is a mutant pneumolysin protein comprising amino acid substitutions at positions 65, 293 and 428 of the wild type sequence.

19. The composition of claim 18 wherein the three amino acid substitutions comprise T₆₅→C, G₂₉₃→C, and C₄₂₈→A.

20. The composition of claim 18 wherein said composition comprises about 5 to 100 μg/dose of said pneumolysin.

21. The composition of claim 1 wherein the composition further comprises an adjuvant optionally selected from the group consisting of aluminum hydroxide, aluminum phosphate, and phosphate treated aluminum hydroxide.

22-23. (canceled)

24. A vaccine comprising the immunogenic composition of claim 1 and a pharmaceutically acceptable excipient.

25. A process for making a vaccine comprising mixing the immunogenic composition of claim 1 with a pharmaceutically acceptable excipient.

26. A method of immunizing a human subject against disease caused by S. pneumoniae infection comprising administrating to the subject an immunologically effective amount of the immunogenic composition of claim 1 wherein, optionally, the human subject is an infant and the disease is at least one disease selected from the group consisting of meningtitis, bacteriaemia, pneumonia, conjunctivitis, otitis media, and invasive pneumococcal disease, wherein the immunization is optionally protective.

27-33. (canceled)

34. The composition of claim 2 further comprising at least one additional antigenic component for conferring protection against disease caused by S. pneumoniae infection.

35-38. (canceled)

39. A method for treating or preventing an infection in a mammal by a Streptococcus bacterial species comprising administering to the mammal a composition selected from the group consisting of:
an effective amount of the immunogenic composition of claim 1;
an antibody which specifically binds to a polypeptide having at least 80% identity to SEQ ID NO:1;
an antibody which specifically binds to a polypeptides having at least 80% identity to SEQ. ID NO:2;
an antibody which specifically binds to a polypeptide having at least 80% identity to SEQ ID NO:1 and an antibody which specifically binds to a polypeptides having at least 80% identity to SEQ ID NO:2;
an antibody which specifically binds to a polypeptide having at least 80% identity to SEQ ID NO:5;
an antibody which specifically binds to a polypeptide having at least 80% identity to SEQ ID NO:7; and,
an antibody which specifically binds to a polypeptide having at least 80% identity to SEQ ID NO:5 and an antibody which specifically binds to a polypeptide having at least 80% identity to SEQ ID NO:7.

40. (canceled)

41. An immunogenic composition of claim 21 comprising an isolated immunogenic S. pneumoniae PcpA polypeptide and/or an isolated immunogenic S. pneumoniae PhtD polypeptide, at least one additional S. pneumoniae polypeptide, and an oil-in-water adjuvant emulsion;
the oil-in-water adjuvant emulsion comprising at least: squalene, an aqueous solvent, a polyoxyethylene alkyl ether hydrophilic nonionic surfactant, and a hydrophobic nonionic surfactant, wherein the emulsion is thermoreversible and wherein 90% of the population by volume of the oil drops has a size less than 200 nm.

42. (canceled)

43. The immunogenic composition of claim 41 wherein the composition further comprises pneumolysin.

44. The immunogenic of claim 43 wherein the pneumolysin is detoxified.

45. The immunogenic composition of claim 44 wherein the pneumolysin has been detoxified genetically.

46-61. (canceled)

62. A composition of claim 1 comprising at least one of a immunogenic PcpA polypeptide, an immunogenic PhtX polypeptide, and/or a detoxified pneumolysin polypeptide; and one or more pharmaceutically acceptable excipients, wherein the one or more pharmaceutically acceptable excipients increases thermal stability of the polypeptide, relative to a composition lacking the one or more pharmaceutically acceptable excipients wherein, optionally,
the one or more pharmaceutically acceptable excipients increases the thermal stability of the polypeptide by 0.5° C. or more, relative to a composition lacking the one or more pharmaceutically acceptable excipients optionally selected from the group consisting of one or more of the excipients listed in Table 11; a buffer optionally selected from the group consisting of Tris-HCL, Tris-HCL with NaCl, and HEPES and is at a concentration of 5-100 mM; tonicity agents; simple carbohydrates; one or more sugars optionally selected from sorbitol, trehalose, and sucrose at a concentration of 1-30%; carbohydrate polymers; amino acids; oligopeptides; polyamino acids; polyhydric alcohols and ethers thereof; detergents; lipids; surfactants; antioxidants; salts; or combinations thereof;
the composition further comprises an adjuvant that is, optionally, an aluminum compound;
the composition is in liquid form; or,
the composition is in dry powder form, freeze dried, spray dried or foam dried.

63-75. (canceled)

76. A method of making a composition comprising an immunogenic PcpA polypeptide and one or more pharmaceutically acceptable excipients, wherein the one or more pharmaceutically acceptable excipients increases thermal stability of the PcpA polypeptide relative to a composition lacking the one or more pharmaceutically acceptable excipients, the method comprising providing an immunogenic PcpA polypeptide and admixing the polypeptide with the one or more pharmaceutically acceptable excipients.

77-80. (canceled)


SEQUENCE LISTING (10 sequences)

SEQ ID NO: 1 - protein, 838 aa, Streptococcus pneumoniae
MKINKKYLAGSVAVLALSVCSYELGRHQAGQVKKESNRVSYIDGDQAGQKAENLTPDEVSKREGINAEQIVIKITDQGYVTSHGDHYHYYNGKVPYDAIISEELLMKDPNYQLKDSDIVNEIKGGYVIKVDGKYYVYLKDAAHADNIRTKEEIKRQKQEHSHNHNSRADNAVAAARAQGRYTTDDGYIFNASDIIEDTGDAYIVPHGDHYHYIPKNELSASELAAAEAYWNGKQGSRPSSSSSYNANPVQPRLSENHNLTVTPTYHQNQGENISSLLRELYAKPLSERHVESDGLIFDPAQITSRTARGVAVPHGNHYHFIPYEQMSELEKRIARIIPLRYRSNHWVPDSRPEQPSPQSTPEPSPSLQPAPNPQPAPSNPIDEKLVKEAVRKVGDGYVFEENGVSRYIPAKDLSAETAAGIDSKLAKQESLSHKLGAKKTDLPSSDREFYNKAYDLLARIHQDLLDNKGRQVDFEVLDNLLERLKDVSSDKVKLVDDILAFLAPIRHPERLGKPNAQITYTDDEIQVAKLAGKYTTEDGYIFDPRDITSDEGDAYVTPHMTHSHWIKKDSLSEAERAAAQAYAKEKGLTPPSTDHQDSGNTEAKGAEAIYNRVKAAKKVPLDRMPYNLQYTVEVKNGSLIIPHYDHYHNIKFEWFDEGLYEAPKGYSLEDLLATVKYYVEHPNERPHSDNGFGNASDHVRKNKADQDSKPDEDKEHDEVSEPTHPESDEKENHAGLNPSADNLYKPSTDTEETEEEAEDTTDEAEIPQVENSVINAKIADAEALLEKVTDPSIRQNAMETLTGLKSSLLLGTKDNNTISAEVDSLLALLKESQPAPIQ

SEQ ID NO: 2 - protein, 641 aa, Streptococcus pneumoniae
MKKTTILSLTTAAVILAAYVPNEPILADTPSSEVIKETKVGSIIQQNNIKYKVLTVEGNIRTVQVGNGVTPVEFEAGQDGKPFTIPTKITVGDKVFTVTEVASQAFSYYPDETGRIVYYPSSITIPSSIKKIQKKGFHGSKAKTIIFDKGSQLEKIEDRAFDFSELEEIELPASLEYIGTSAFSFSQKLKKLTFSSSSKLELISHEAFANLSNLEKLTLPKSVKTLGSNLFRLTTSLKHVDVEEGNESFASVDGVLFSKDKTQLIYYPSQKNDESYKTPKETKELASYSFNKNSYLKKLELNEGLEKIGTFAFADAIKLEEISLPNSLETIERLAFYGNLELKELILPDNVKNFGKHVMNGLPKLKSLTIGNNINSLPSFFLSGVLDSLKEIHIKNKSTEFSVKKDTFAIPETVKFYVTSEHIKDVLKSNLSTSNDIIVEKVDNIKQETDVAKPKKNSNQGVVGWVKDKGLWYYLNESGSMATGWVKDKGLWYYLNESGSMATGWVKDKGLWYYLNESGSMATGWVKDKGLWYYLNESGSMATGWVKDKGLWYYLNESGSMATGWVKDKGLWYYLNESGSMATGWVKDKGLWYYLNESGSMATGWFTVSGKWYYTYNSGDLLVNTTTPDGYRVNANGEWVG

SEQ ID NO: 3 - DNA, 2514 bp, Streptococcus pneumoniae
atgaaaatcaataaaaaatatctagcaggttcagtggcagtccttgccctaagtgtttgttcctatgaacttggtcgtcaccaagctggtcaggttaagaaagagtctaatcgagtttcttatatagatggtgatcaggctggtcaaaaggcagaaaatttgacaccagatgaagtcagtaagagagaggggatcaacgccgaacaaattgttatcaagattacggatcaaggttatgtgacctctcatggagaccattatcattactataatggcaaggttccttatgatgccatcatcagtgaagaacttctcatgaaagatccgaattatcagttgaaggattcagacattgtcaatgaaatcaagggtggctatgtgattaaggtagacggaaaatactatgtttaccttaaagatgcggcccatgcggacaatattcggacaaaagaagagattaaacgtcagaagcaggaacacagtcataatcataactcaagagcagataatgctgttgctgcagccagagcccaaggacgttatacaacggatgatgggtatatcttcaatgcatctgatatcattgaggacacgggtgatgcttatatcgttcctcacggcgaccattaccattacattcctaagaatgagttatcagctagcgagttagctgctgcagaagcctattggaatgggaagcagggatctcgtccttcttcaagttctagttataatgcaaatccagttcaaccaagattgtcagagaaccacaatctgactgtcactccaacttatcatcaaaatcaaggggaaaacatttcaagccttttacgtgaattgtatgctaaacccttatcagaacgccatgtagaatctgatggccttattttcgacccagcgcaaatcacaagtcgaaccgccagaggtgtagctgtccctcatggtaaccattaccactttatcccttatgaacaaatgtctgaattggaaaaacgaattgctcgtattattccccttcgttatcgttcaaaccattgggtaccagattcaagaccagaacaaccaagtccacaatcgactccggaacctagtccaagtctgcaacctgcaccaaatcctcaaccagctccaagcaatccaattgatgagaaattggtcaaagaagctgttcgaaaagtaggcgatggttatgtctttgaggagaatggagtttctcgttatatcccagccaaggatctttcagcagaaacagcagcaggcattgatagcaaactggccaagcaggaaagtttatctcataagctaggagctaagaaaactgacctcccatctagtgatcgagaattttacaataaggcttatgacttactagcaagaattcaccaagatttacttgataataaaggtcgacaagttgattttgaggttttggataacctgttggaacgactcaaggatgtctcaagtgataaagtcaagttagtggatgatattcttgccttcttagctccgattcgtcatccagaacgtttaggaaaaccaaatgcgcaaattacctacactgatgatgagattcaagtagccaagttggcaggcaagtacacaacagaagacggttatatctttgatcctcgtgatataaccagtgatgagggggatgcctatgtaactccacatatgacccatagccactggattaaaaaagatagtttgtctgaagctgagagagcggcagcccaggcttatgctaaagagaaaggtttgacccctccttcgacagaccatcaggattcaggaaatactgaggcaaaaggagcagaagctatctacaaccgcgtgaaagcagctaagaaggtgccacttgatcgtatgccttacaatcttcaatatactgtagaagtcaaaaacggtagtttaatcatacctcattatgaccattaccataacatcaaatttgagtggtttgacgaaggcctttatgaggcacctaaggggtatagtcttgaggatcttttggcgactgtcaagtactatgtcgaacatccaaacgaacgtccgcattcagataatggttttggtaacgctagtgaccatgttcgtaaaaataaggcagaccaagatagtaaacctgatgaagataaggaacatgatgaagtaagtgagccaactcaccctgaatctgatgaaaaagagaatcacgctggtttaaatccttcagcagataatctttataaaccaagcactgatacggaagagacagaggaagaagctgaagataccacagatgaggctgaaattcctcaagtagagaattctgttattaacgctaagatagcagatgcggaggccttgctagaaaaagtaacagatcctagtattagacaaaatgctatggagacattgactggtctaaaaagtagtcttcttctcggaacgaaagataataacactatttcagcagaagtagatagtctcttggctttgttaaaagaaagtcaaccggctcctatacag

SEQ ID NO: 4 - DNA, 1923 bp, Streptococcus pneumoniae
atgaaaaaaactacaatattatcattaactacagctgcggttattttagcagcatatgtccctaatgaaccaatcctagcagatactcctagttcggaagtaatcaaagagactaaagttggaagtattattcaacaaaataatatcaaatataaggttctaactgtagaaggtaacataagaactgttcaagtgggtaatggagttactcctgtagagtttgaagctggtcaagatggaaaaccattcacgattcctacaaaaatcacagtaggtgataaagtatttaccgttactgaagtagctagtcaagcttttagttattatccagatgaaacaggtagaattgtctactatcctagctctattactatcccatcaagcataaaaaaaatacaaaaaaaaggcttccatggaagtaaagctaaaactattatttttgacaaaggcagtcagctggagaaaattgaagatagagcttttgatttttctgaattagaagagattgaattgcctgcatctctagaatatattggaacaagtgcattttcttttagtcaaaaattgaaaaagctaaccttttcctcaagttcaaaattagaattaatatcacatgaggcttttgctaatttatcaaatttagagaaactaacattaccaaaatcggttaaaacattaggaagtaatctatttagactcactactagcttaaaacatgttgatgttgaagaaggaaatgaatcgtttgcctcagttgatggtgttttgttttcaaaagataaaactcaattaatttattatccaagtcaaaaaaatgacgaaagttataaaacgcctaaggagacaaaagaacttgcatcatattcgtttaataaaaattcttacttgaaaaaactcgaattgaatgaaggtttagaaaaaatcggtacttttgcatttgcggatgcgattaaacttgaagaaattagcttaccaaatagtttagaaactattgaacgtttagccttttacggtaatttagaattaaaagaacttatattaccagataatgttaaaaattttggtaaacacgttatgaacggtttaccaaaattaaaaagtttaacaattggtaataatatcaactcattgccgtccttcttcctaagtggcgtcttagattcattaaaggaaattcatattaagaataaaagtacagagttttctgtgaaaaaagatacatttgcaattcctgaaactgttaagttctatgtaacatcagaacatataaaagatgttcttaaatcaaatttatctactagtaatgatatcattgttgaaaaagtagataatataaaacaagaaactgatgtagctaaacctaaaaagaattctaatcagggagtagttggttgggttaaagacaaaggtttatggtattacttaaacgaatcaggttcaatggctactggttgggttaaagacaaaggtttatggtattacttaaacgaatcaggttcaatggctactggttgggttaaagacaaaggcttatggtattacttaaacgaatcaggttcaatggctactggttgggttaaagacaaaggcttatggtattacttaaatgaatcaggttcaatggctactggttgggttaaagacaaaggcttatggtattacttaaacgaatcaggttcaatggctactggttgggttaaagacaaaggcttatggtattacttaaacgaatcaggttcaatggctactggttgggttaaagacaaaggcttatggtattacttaaatgaatcaggttcaatggctactggttggtttacagtttctggtaaatggtactatacctataattcaggagatttattagtaaacacgactacacccgatggctatcgagtcaatgctaacggtgagtgggtagga

SEQ ID NO: 5 - protein, 820 aa, Streptococcus pneumoniae
MGSYELGRHQAGQVKKESNRVSYIDGDQAGQKAENLTPDEVSKREGINAEQIVIKITDQGYVTSHGDHYHYYNGKVPYDAIISEELLMKDPNYQLKDSDIVNEIKGGYVIKVDGKYYVYLKDAAHADNIRTKEEIKRQKQEHSHNHNSRADNAVAAARAQGRYTTDDGYIFNASDIIEDTGDAYIVPHGDHYHYIPKNELSASELAAAEAYWNGKQGSRPSSSSSYNANPVQPRLSENHNLTVTPTYHQNQGENISSLLRELYAKPLSERHVESDGLIFDPAQITSRTARGVAVPHGNHYHFIPYEQMSELEKRIARIIPLRYRSNHWVPDSRPEQPSPQSTPEPSPSLQPAPNPQPAPSNPIDEKLVKEAVRKVGDGYVFEENGVSRYIPAKDLSAETAAGIDSKLAKQESLSHKLGAKKTDLPSSDREFYNKAYDLLARIHQDLLDNKGRQVDFEVLDNLLERLKDVSSDKVKLVDDILAFLAPIRHPERLGKPNAQITYTDDEIQVAKLAGKYTTEDGYIFDPRDITSDEGDAYVTPHMTHSHWIKKDSLSEAERAAAQAYAKEKGLTPPSTDHQDSGNTEAKGAEAIYNRVKAAKKVPLDRMPYNLQYTVEVKNGSLIIPHYDHYHNIKFEWFDEGLYEAPKGYSLEDLLATVKYYVEHPNERPHSDNGFGNASDHVRKNKADQDSKPDEDKEHDEVSEPTHPESDEKENHAGLNPSADNLYKPSTDTEETEEEAEDTTDEAEIPQVENSVINAKIADAEALLEKVTDPSIRQNAMETLTGLKSSLLLGTKDNNTISAEVDSLLALLKESQPAPIQ

SEQ ID NO: 6 - DNA, 2463 bp, Streptococcus pneumoniae
atgggatcctatgaacttggtcgtcaccaagctggtcaggttaagaaagagtctaatcgagtttcttatatagatggtgatcaggctggtcaaaaggcagaaaatttgacaccagatgaagtcagtaagagagaggggatcaacgccgaacaaattgttatcaagattacggatcaaggttatgtgacctctcatggagaccattatcattactataatggcaaggttccttatgatgccatcatcagtgaagaacttctcatgaaagatccgaattatcagttgaaggattcagacattgtcaatgaaatcaagggtggctatgtgattaaggtagacggaaaatactatgtttaccttaaagatgcggcccatgcggacaatattcggacaaaagaagagattaaacgtcagaagcaggaacacagtcataatcataactcaagagcagataatgctgttgctgcagccagagcccaaggacgttatacaacggatgatgggtatatcttcaatgcatctgatatcattgaggacacgggtgatgcttatatcgttcctcacggcgaccattaccattacattcctaagaatgagttatcagctagcgagttagctgctgcagaagcctattggaatgggaagcagggatctcgtccttcttcaagttctagttataatgcaaatccagttcaaccaagattgtcagagaaccacaatctgactgtcactccaacttatcatcaaaatcaaggggaaaacatttcaagccttttacgtgaattgtatgctaaacccttatcagaacgccatgtagaatctgatggccttattttcgacccagcgcaaatcacaagtcgaaccgccagaggtgtagctgtccctcatggtaaccattaccactttatcccttatgaacaaatgtctgaattggaaaaacgaattgctcgtattattccccttcgttatcgttcaaaccattgggtaccagattcaagaccagaacaaccaagtccacaatcgactccggaacctagtccaagtctgcaacctgcaccaaatcctcaaccagctccaagcaatccaattgatgagaaattggtcaaagaagctgttcgaaaagtaggcgatggttatgtctttgaggagaatggagtttctcgttatatcccagccaaggatctttcagcagaaacagcagcaggcattgatagcaaactggccaagcaggaaagtttatctcataagctaggagctaagaaaactgacctcccatctagtgatcgagaattttacaataaggcttatgacttactagcaagaattcaccaagatttacttgataataaaggtcgacaagttgattttgaggttttggataacctgttggaacgactcaaggatgtctcaagtgataaagtcaagttagtggatgatattcttgccttcttagctccgattcgtcatccagaacgtttaggaaaaccaaatgcgcaaattacctacactgatgatgagattcaagtagccaagttggcaggcaagtacacaacagaagacggttatatctttgatcctcgtgatataaccagtgatgagggggatgcctatgtaactccacatatgacccatagccactggattaaaaaagatagtttgtctgaagctgagagagcggcagcccaggcttatgctaaagagaaaggtttgacccctccttcgacagaccatcaggattcaggaaatactgaggcaaaaggagcagaagctatctacaaccgcgtgaaagcagctaagaaggtgccacttgatcgtatgccttacaatcttcaatatactgtagaagtcaaaaacggtagtttaatcatacctcattatgaccattaccataacatcaaatttgagtggtttgacgaaggcctttatgaggcacctaaggggtatagtcttgaggatcttttggcgactgtcaagtactatgtcgaacatccaaacgaacgtccgcattcagataatggttttggtaacgctagtgaccatgttcgtaaaaataaggcagaccaagatagtaaacctgatgaagataaggaacatgatgaagtaagtgagccaactcaccctgaatctgatgaaaaagagaatcacgctggtttaaatccttcagcagataatctttataaaccaagcactgatacggaagagacagaggaagaagctgaagataccacagatgaggctgaaattcctcaagtagagaattctgttattaacgctaagatagcagatgcggaggccttgctagaaaaagtaacagatcctagtattagacaaaatgctatggagacattgactggtctaaaaagtagtcttcttctcggaacgaaagataataacactatttcagcagaagtagatagtctcttggctttgttaaaagaaagtcaaccggctcctatacagtag

SEQ ID NO: 7 - protein, 445 aa, Streptococcus pneumoniae
MADTPSSEVIKETKVGSIIQQNNIKYKVLTVEGNIGTVQVGNGVTPVEFEAGQDGKPFTIPTKITVGDKVFTVTEVASQAFSYYPDETGRIVYYPSSITIPSSIKKIQKKGFHGSKAKTIIFDKGSQLEKIEDRAFDFSELEEIELPASLEYIGTSAFSFSQKLKKLTFSSSSKLELISHEAFANLSNLEKLTLPKSVKTLGSNLFRLTTSLKHVDVEEGNESFASVDGVLFSKDKTQLIYYPSQKNDESYKTPKETKELASYSFNKNSYLKKLELNEGLEKIGTFAFADAIKLEEISLPNSLETIERLAFYGNLELKELILPDNVKNFGKHVMNGLPKLKSLTIGNNINSLPSFFLSGVLDSLKEIHIKNKSTEFSVKKDTFAIPETVKFYVTSEHIKDVLKSNLSTSNDIIVEKVDNIKQETDVAKPKKNSNQGVVGWVKDKG

SEQ ID NO: 8 - DNA, 1338 bp, Streptococcus pneumoniae
atggcagatactcctagttcggaagtaatcaaagagactaaagttggaagtattattcaacaaaataatatcaaatataaggttctaactgtagaaggtaacataggaactgttcaagtgggtaatggagttactcctgtagagtttgaagctggtcaagatggaaaaccattcacgattcctacaaaaatcacagtaggtgataaagtatttaccgttactgaagtagctagtcaagcttttagttattatccagatgaaacaggtagaattgtctactatcctagctctattactatcccatcaagcataaaaaaaatacaaaaaaaaggcttccatggaagtaaagctaaaactattatttttgacaaaggcagtcagctggagaaaattgaagatagagcttttgatttttctgaattagaagagattgaattgcctgcatctctagaatatattggaacaagtgcattttcttttagtcaaaaattgaaaaagctaaccttttcctcaagttcaaaattagaattaatatcacatgaggcttttgctaatttatcaaatttagagaaactaacattaccaaaatcggttaaaacattaggaagtaatctatttagactcactactagcttaaaacatgttgatgttgaagaaggaaatgaatcgtttgcctcagttgatggtgttttgttttcaaaagataaaactcaattaatttattatccaagtcaaaaaaatgacgaaagttataaaacgcctaaggagacaaaagaacttgcatcatattcgtttaataaaaattcttacttgaaaaaactcgaattgaatgaaggtttagaaaaaatcggtacttttgcatttgcggatgcgattaaacttgaagaaattagcttaccaaatagtttagaaactattgaacgtttagccttttacggtaatttagaattaaaagaacttatattaccagataatgttaaaaattttggtaaacacgttatgaacggtttaccaaaattaaaaagtttaacaattggtaataatatcaactcattgccgtccttcttcctaagtggcgtcttagattcattaaaggaaattcatattaagaataaaagtacagagttttctgtgaaaaaagatacatttgcaattcctgaaactgttaagttctatgtaacatcagaacatataaaagatgttcttaaatcaaatttatctactagtaatgatatcattgttgaaaaagtagataatataaaacaagaaactgatgtagctaaacctaaaaagaattctaatcagggagtagttggttgggttaaagacaaaggttaa

SEQ ID NO: 9 - protein, 471 aa, Streptococcus pneumoniae
MANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIREATGLAWEWWRTVYEKTDLPLVRKRTISIWGTTLYPQVEDKVEND

SEQ ID NO: 10 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND